(19) United States
(12) Patent Application Publication
(10) Pub. No.: US 2005/0232937 A1
(43) Pub. Date: Oct. 20, 2005

(54) Paramycobacterial diagnostics and vaccines

(76) Inventors: Petrus Theodorus Johannes Willemsen, Amsterdam (NL); Sjoukje Fetsje Westerveen, Leeuwarden (NL); Douwe Bakker, Am Lelystad (NL); Fred Govert van Zijderveld, Dc Lelystad (NL); Jelle Egbertus Rudolfus Thole, Br Dronten (NL)

Correspondence Address: INTERVET U.S.;PATENT DEPARTMENT, PO BOX 318, MILLSBORO, DE, 19966-0318, US

(21) Appl. No.: 10/501,127

(22) PCT Filed: Jan. 13, 2003
(86) PCT No.: PCT/NL03/00020

§ 371 Date: Sep. 10, 2004

(30) Foreign Application Priority Data
Jan. 11, 2002 (EP) 02075089.9

(51) Int. Cl. A61K 39/02; C07K 14/35; C12N 1/21; C07H 21/04; C12N 15/74

(52) U.S. Cl. 424/190.1; 435/69.3; 435/252.3; 435/471; 530/350; 536/23.7

(57) ABSTRACT

The present invention relates to nucleic acid sequences encoding Mycobacterium avium subspecies paratuberculosis proteins, to parts of such nucleic acid sequences that encode an immunogenic fragment of such proteins, to DNA fragments, recombinant DNA molecules, live recombinant carriers and host cells comprising such nucleic acid sequences or such parts thereof. The invention also relates to Mycobacterium avium subspecies paratuberculosis proteins and immunogenic parts thereof encoded by such sequences. Furthermore, the present invention relates to vaccines comprising such nucleic acid sequences and parts thereof, DNA fragments, recombinant DNA molecules, live recombinant carriers and host cells comprising such nucleic acid sequences or such parts thereof, proteins or immunogenic parts thereof and antibodies against such proteins or immunogenic parts thereof. Also, the invention relates to the use of said proteins in vaccines and for the manufacture of vaccines. Moreover, the inventions relates to the use of said nucleic acid sequences, proteins, or antibodies for diagnostic or vaccination purposes. Also, the invention relates to methods for the preparation of such vaccines. Finally the invention relates to diagnostic kits comprising such nucleic acids, proteins or antibodies against such proteins.

The present invention relates to nucleic acid sequences encoding Mycobacterium avium subspecies paratuberculosis proteins, to parts of such nucleic acid sequences that encode an immunogenic fragment of such proteins, to DNA fragments, recombinant DNA molecules, live recombinant carriers and host cells comprising such nucleic acid sequences or such parts thereof. The invention also relates to Mycobacterium avium subspecies paratuberculosis proteins and immunogenic parts thereof encoded by such sequences. Furthermore, the present invention relates to vaccines comprising such nucleic acid sequences and parts thereof DNA fragments, recombinant DNA molecules, live recombinant carriers and host cells comprising such nucleic acid sequences or such parts thereof, proteins or immunogenic parts thereof and antibodies against such proteins or immunogenic parts thereof. Also, the invention relates to the use of said proteins in vaccines and for the manufacture of vaccines. Moreover, the invention relates to the use of said nucleic acid sequences, proteins or antibodies for diagnostic or vaccination purposes. Also, the invention relates to methods for the preparation of such vaccines. Finally the invention relates to diagnostic kits comprising such nucleic acids, proteins or antibodies against such proteins.

Bacteria of the genus Mycobacterium are Gram-positive acid-fast organisms. The genus includes a number of significant human and animal pathogens. Amongst these is M. avium subspecies paratuberculosis, the causative agent of paratuberculosis or Johne's disease, a chronic granulomatous infection leading to disease in ruminants that is currently responsible for very substantial worldwide economic losses in both meat and dairy industry. A large proportion of herds (between 21-70%) are infected worldwide. In Europe, the estimated yearly losses are about GBP 207/diary cow. In the USA, the estimated yearly losses are about 1.5 billion US-dollars. (Harris, N. B. and Barletta, R. G., Clinical Microbiology Reviews 14: 489-512 (2001)).

In addition to its evident pathogenicity in ruminants, M. avium subspecies paratuberculosis is suspected to be the cause of Crohn's disease, a non-specific chronic transmural inflammatory disease of humans that most commonly affects the distal ileum and colon but that may also occur in any part of the gastrointestinal tract from the mouth to the anus and perianal area. (P. Quirke, Gut 2001, 49: 755-760 (2001)), (“Possible links between Crohn’s disease and Paratuberculosis”, European Commission, D-G Health and Consumer Protection, Report of the Scientific Committee on Animal Health and Animal Welfare, 21 Mar. 2000). One of the main problems of Crohn's disease is the fact that there is no cure for the disease. The disease status, once gathered, remains present life-long, causing significant morbidity.

The presence of unexpectedly more thermally tolerant strains of M. avium subspecies paratuberculosis in pasteurised milk in combination with its suspected role in the development of Crohn's disease has raised increasing concern regarding its potential health effects on the human population as well. This recent and unexpected finding has been described by Sung, N. and Collins, M. T. in Appl. Environm. Microbiol. 64: 999-1005 (1998).

Increased awareness of the problem has resulted in renewed urgency for the development of effective diagnostics and vaccines for control and eradication of paratuberculosis.

Mycobacterium avium comprises a large group of mycobacteria that can be divided into three subspecies, M. avium subspecies avium, M. avium subspecies silvaticurn and M. avium subspecies paratuberculosis. M. avium subspecies avium is widely distributed in the natural environment including soil and apparently healthy animals, as well as in birds and humans. M. avium subspecies avium isolates are opportunistic pathogens and generally cause infection and disease in immunocompromised hosts. The complete genomic sequence of M. avium subspecies avium strain 104 is currently being determined. M. avium subspecies siluaticum can produce a disease that resembles paratuberculosis in deer. Although most ruminants are infected with M. avium subspecies paratuberculosis before six months of age, clinical disease generally occurs only after at least two years of age, or later. During this period, bacteria are believed to survive inside host cells, but extracellular episodes of infection in the lumen of the gastrointestinal tract—during which the bacterium becomes detectable in faeces—do also occur (with increasing frequency at later stages of infection). Currently available (immuno-) diagnostics against M. avium subspecies paratuberculosis have a relatively poor sensitivity, especially with respect to the detection of early or latent infection, and therefore are not effective as a tool for disease control. Whole cell Mycobacterial vaccines that are to some measure thought to be effective in freeing herds from clinical disease are available, but these vaccines essentially interfere with the immunodiagnosis of bovine tuberculosis and do not inhibit transmission of disease. To date several antigenic components of M. avium subspecies paratuberculosis have been identified. The antigenic molecules of M. avium subspecies paratuberculosis described previously comprise glycolipids and protein antigens identified with essentially monospecific early sera raised in small experimental animals. The cell wall glycolipid molecule lipoarabinomannan (LAM) was identified by its recognition by monoclonal antibodies raised against cell filtrate released by the bacterium, and has subsequently been purified and used for the development of a serodiagnostic ELISA (Mutharia et al., Infect. Immun. 1997.65:387-394; Jark et al., 1997. Vet. Microbiol. 57:189-198). In addition, protein antigens with molecular weight of 14 kD (Olsen et al. Clin. Diagn. Lab. Immunol. 2001.8:797-801), 18 kD (bacterioferritin; Elsaghier et al. Clin. Exp. Immunol. 1992 90:503-508), 19 kD (AhpD; Olsen et al., Infect. Immun. 2000.68:801-808), 24 kD (p24BCD; Elsaghier et al. Clin. Exp. Immunol. 1992 90:503-508), 30 kD (p30; Burrels et al.; Vet. Immunol. Immunpathol. 1995. 45:311-320), 34 kD (Gilot et al. J. Bact. 1993. 175:4930-4935; De Kesel et al J. Clin. Microbiol. 1993. 31: 947-954; Coetsier et al., Clin. Diagn. Lab. Immunol. 1998.5: 446-451), 34.5 kD(Mutharia et al., Infect. Immun. 1997.65:387-394), ), a 35 kD protein Dheenadhayalan and Chang, unpublished data), 38 kD (Elsaghier et al. Clin. Exp. Immunol. 199290:503-508), 44.3 kD (Mutharia et al., Infect. Immun. 1997.65:387-394), 45 kD (AhpC; Olsen et al., Infect. Immun. 2000. 68:801-808), 65 kD (hsp65; Koets et al., Vet. Immunol. Immunopathol. 1999. 70:105-115), 70 kD (hsp70; Stevenson et al., 1991. Nucleic Acids Res. 19:4552; Koets et al., Vet. Immunol. Immunopathol. 1999.70:105-115), and a superoxide dismutase molecule (Mullerad et al., FEMS Immunol. Med. Microbiol 34: 81 (2002)) have been identified and (artly) characterized. Only few of these have been evaluated for the development of diagnostics or vaccines (34 kD; Coetsier et al., Clin. Diagn. Lab. Immunol. 1998. 5: 446-451). Current diagnostics and vaccines are therefore still based on rather crude antigenic materials. The lipoarabinomannan (Mutharia et al., Infect. Immun. 1997. 65:387-394; Jark et al., 1997. Vet. Microbiol. 57:189-198) and 34 kD antigen (Gilot et al. J. Bact. 1993. 175:4930-4935; De Kesel et al J. Clin. Microbiol. 1993. 31: 947-954; Coetsier et al., Clin. Diagn. Lab. Immunol. 1998.5: 446-451) have been described in DE19621488 and WO9216628, for use in diagnosis and vaccines. Several other molecules have been submitted for use in diagnostics, vaccines and therapeutics. These are proteins encoded on insertion sequence ISM-1 (EP0288306 and U.S. Pat. No. 5,225,324;), the mycobacterial DAP molecule (U.S. Pat. No. 9,523,226), a 36 kD antigen (U.S. Pat. No. 5,776,692), a soluble antigen preparation (RU2118538), an extra cellular protein with an iron-reducing capacity (DE 19728834), and an acylase (WO9949054).

It is an objective of the present invention to provide polypeptides that are capable of contributing to protection against the pathogenic effects of Mycobacterium avium subspecies paratuberculosis infection in mammals, more specifically humans and cattle. Moreover, a number of these polypeptides and antibodies against these polypeptides provide efficient diagnostic tools.

It was now first of all surprisingly found that nine different polypeptides could be specifically identified in expression libraries and isolated, and two additional polypeptides could be identified in proteomics, each of these different polypeptides being capable of inducing an immune response against Mycobacterium avium subspecies paratuberculosis and suitable as vaccine components.

The inventors have found that these polypeptides can be used, either alone or in combination with each other, as vaccine components to provide a vaccine which indeed contributes to the protection against Mycobacterium avium subspecies paratuberculosis infection in mammals, more specifically in humans and cattle and helps to decrease the damage caused by Mycobacterium avium subspecies paratuberculosis.

Three different approaches have been used for the detection of the (genes encoding the) vaccine components and diagnostic tools according to the invention. These approaches are presented in more detail in the Examples. One approach uses a very specific antiserum for the detection of genes encoding immunoreactive Mycobacterium avium subspecies paratuberculosis proteins in an expression library. The antiserum used differs from antisera commonly used for the screening of expression libraries in the sense that is has been obtained from cows that have been infected with Mycobacterium avium subspecies paratuberculosis for a very prolonged period. Furthermore, these antisera were taken from cows that were found to be naturally infected with M. avium subsp. paratuberculosis, but had no history of infection with tuberculosis, brucellosis or leucosis. This was evidenced by finding at least two M. avium subsp. paratuberculosis positive faeces samples within an approximately two year long period before obtaining the test-serum for use in screening, and finding essentially no antibodies or other immune responses directed against agents causing tuberculosis that are cross-reactive with paratuberculosis antigens. Sera thus obtained are very useful in immunoscreening for M. avium subsp. paratuberculosis antigens, since they are very broadly reactive against relevant M. avium subsp. paratuberculosis peptide fragments whereas on the other hand they show essentially no or only little specific reactivity with the pathogens causing tuberculosis, brucellosis or leucosis.

This approach has led to the finding of three novel immunogenic proteins for which the coding sequences are depicted in SEQ ID NO: 1, 3, and 5 as given below.

The gene encoding the first protein has now been cloned and sequenced and a nucleic acid sequence of the gene that comprises immunogenic determinants is depicted in SEQ ID NO: 1. The full-length gene encodes a protein (as depicted in SEQ ID NO: 2) with a molecular mass of 28 kD.

It is well-known in the art, that many different nucleic acid sequences can encode one and the same protein. This phenomenon is commonly known as wobble in the second and especially the third base of each triplet encoding an amino acid. This phenomenon can result in a heterology for two nucleic acid sequences still encoding the same protein. Therefore, in principle, two nucleic acid sequences having a sequence homology as low as 70% can still encode one and the same protein.

Thus, one form of a first embodiment of the present invention relates to a nucleic acid sequence encoding an Mycobacterium avium subspecies paratuberculosis protein or a part of said nucleic acid sequence that encodes an immunogenic fragment of said protein wherein said nucleic acid sequence or said part thereof has at least 85% homology with the nucleic acid sequence of the Mycobacterium avium subspecies paratuberculosis protein gene as depicted in SEQ ID NO: 1.

The concept of immunogenic fragment is defined below. The length of a nucleic acid sequence encoding an immunogenic fragment is usually at least 18 or more often 21 nucleotides, but preferably 24, 27, 30, 33 or even 36 nucleotides.

The molecular weight of all proteins according to the invention when determined in gel electrophoresis on a polyacryl amide gel may vary to a certain extend, due to slight variability of molecular weight determination frequently encountered in the art. Therefore the molecular weight of the proteins according to the invention should be interpreted as to be its theoretical molecular weight +/−5 kD.

Preferably, a nucleic acid sequence according to the invention encoding this Mycobacterium avium subspecies paratuberculosis protein or a part of that nucleic acid sequence that encodes an immunogenic fragment of that protein has at least 90%, preferably 93%, more preferably 95% homology with the nucleic acid sequence of the Mycobacterium avium subspecies paratuberculosis protein gene as depicted in SEQ ID NO: 1.

Even more preferred is a homology level of 98%, 99% or even 100%.

The level of nucleotide homology can be determined with the computer program “BLAST 2 SEQUENCES” by selecting sub-program: “BLASTN” that can be found at www.ncbi.nlm.nih.govblast/bl2seg/bl2.html.

A reference for this program is Tatiana A Tatusova, Thomas L. Madden FEMS Microbiol. Letters 174: 247-250 (1999). Parameters used are the default parameters:

Reward for a match: +1. Penalty for a mismatch: −2. Open gap: 5. Extension gap: 2. Gap x_dropoff: 50.

Nucleotide sequences that are complementary to the sequence depicted in SEQ ID NO 1 or any of SEQ ID NO 3, 5, 7, 9, 11, 13, 15 or 17 that will be described below, or nucleotide sequences that comprise tandem arrays of the sequences according to the invention, are also within the scope of the invention.

Another form of this embodiment relates to a nucleic acid sequence encoding a 14 kD Mycobacterium avium subspecies paratuberculosis protein or a part of said nucleic acid sequence that encodes an immunogenic fragment of said protein wherein said nucleic acid sequence or said part thereof has at least 85% homology with the nucleic acid sequence of the Mycobacterium avium subspecies parotuberculosis protein gene as depicted in SEQ ID NO: 3.

Preferably, a nucleic acid sequence according to the invention encoding this 14 kD Mycobacterium avium subspecies paratuberculosis protein or a part of that nucleic acid sequence that encodes an immunogenic fragment of that protein has at least 90%, preferably 93%, more preferably 95% homology with the nucleic acid sequence of the Mycobacterium avium subspecies paratuberculosis protein gene as depicted in SEQ ID NO: 3.

Even more preferred is a homology level of 98%, 99% or even 100%.

Still another form of this embodiment relates to a nucleic acid sequence encoding a 9 kD Mycobacterium avium subspecies paratuberculosis protein or a part of said nucleic acid sequence that encodes an immunogenic fragment of said protein wherein said nucleic acid sequence or said part thereof has at least 85% homology with the nucleic acid sequence of the Mycobacterium avium subspecies paratuberculosis protein gene as depicted in SEQ ID NO: 5.

Preferably, a nucleic acid sequence according to the invention encoding this 9 kD Mycobacterium avium subspecies paratuberculosis protein or a part of that nucleic acid sequence that encodes an immunogenic fragment of that protein has at least 90%, preferably 93%, more preferably 95% homology with the nucleic acid sequence of the Mycobacterium avium subspecies paratuberculosis protein gene as depicted in SEQ ID NO: 5.

Even more preferred is a homology level of 98%, 99% or even 100%.

Another approach used for the detection of immunologically important polypeptides was based upon the use of highly specific monoclonal antibodies against Mycobacterium avium subspecies paratuberculosis proteins. This approach has the advantage that it is even more specific than the approach using the specific antisera against Mycobacterium avium subspecies paratuberculosis described above.

Use of these monoclonal antibodies led to the identification and isolation of six additional immunogenic Mycobacterium avium subspecies paratuberculosis proteins.

Therefore, again another form of this embodiment relates to a nucleic acid sequence encoding a 47 kD Mycobacterium avium subspecies paratuberculosis protein or a part of said nucleic acid sequence that encodes an immunogenic fragment of said protein wherein said nucleic acid sequence or said part thereof has at least 85% homology with the nucleic acid sequence of the Mycobacterium avium subspecies paratuberculosis protein gene as depicted in SEQ ID NO: 7.

Preferably, a nucleic acid sequence according to the invention encoding this 47 kD Mycobacterium avium subspecies paratuberculosis protein or a part of that nucleic acid sequence that encodes an immunogenic fragment of that protein has at least 90%, preferably 93%, more preferably 95% homology with the nucleic acid sequence of the Mycobacterium avium subspecies paratuberculosis protein gene as depicted in SEQ ID NO: 7.

Even more preferred is a homology level of 98%, 99% or even 100%.

Again another form of this embodiment relates to a nucleic acid sequence encoding a Mycobacterium avium subspecies paratuberculosis protein or a part of said nucleic acid sequence that encodes an immunogenic fragment of said protein wherein said nucleic acid sequence or said part thereof has at least 85% homology with the nucleic acid sequence of the Mycobacterium avium subspecies paratuberculosis protein gene as depicted in SEQ ID NO: 9.

Preferably, a nucleic acid sequence according to the invention encoding this Mycobacterium avium subspecies paratuberculosis protein or a part of that nucleic acid sequence that encodes an immunogenic fragment of that protein has at least 90%, preferably 93%, more preferably 95% homology with the nucleic acid sequence of the Mycobacterium avium subspecies paratuberculosis protein gene as depicted in SEQ ID NO: 9.

Even more preferred is a homology level of 98%, 99% or even 100%.

Another form of this embodiment relates to a nucleic acid sequence encoding a Mycobacterium avium subspecies paratuberculosis protein or a part of said nucleic acid sequence that encodes an immunogenic fragment of said protein wherein said nucleic acid sequence or said part thereof has at least 85% homology with the nucleic acid sequence of the Mycobacterium avium subspecies paratuberculosis protein gene as depicted in SEQ ID NO: 11.

Preferably, a nucleic acid sequence according to the invention encoding this Mycobacterium avium subspecies paratuberculosis protein or a part of that nucleic acid sequence that encodes an immunogenic fragment of that protein has at least 90%, preferably 93%, more preferably 95% homology with the nucleic acid sequence of the Mycobacterium avium subspecies paratuberculosis protein gene as depicted in SEQ ID NO: 11.

Even more preferred is a homology level of 98%, 99% or even 100%.

Again another form of this embodiment relates to a nucleic acid sequence encoding a Mycobacterium avium subspecies paratuberculosis protein or a part of said nucleic acid sequence that encodes an immunogenic fragment of said protein wherein said nucleic acid sequence or said part thereof has at least 85% homology with the nucleic acid sequence of the Mycobacterium avium subspecies paratuberculosis protein gene as depicted in SEQ ID NO: 13.

Preferably, a nucleic acid sequence according to the invention encoding this Mycobacterium avium subspecies paratuberculosis protein or a part of that nucleic acid sequence that encodes an immunogenic fragment of that protein has at least 90%, preferably 93%, more preferably 95% homology with the nucleic acid sequence of the Mycobacterium avium subspecies paratuberculosis protein gene as depicted in SEQ ID NO: 13.

Even more preferred is a homology level of 98%, 99% or even 100%.

Again another form of this embodiment relates to a nucleic acid sequence encoding a Mycobacterium avium subspecies paratuberculosis protein or a part of said nucleic acid sequence that encodes an immunogenic fragment of said protein wherein said nucleic acid sequence or said part thereof has at least 85% homology with the nucleic acid sequence of the Mycobacterium avium subspecies paratuberculosis protein gene as depicted in SEQ ID NO: 15.

Preferably, a nucleic acid sequence according to the invention encoding this Mycobacterium avium subspecies paratuberculosis protein or a part of that nucleic acid sequence that encodes an immunogenic fragment of that protein has at least 90%, preferably 93%, more preferably 95% homology with the nucleic acid sequence of the Mycobacterium avium subspecies paratuberculosis protein gene as depicted in SEQ ID NO: 15.

Even more preferred is a homology level of 98%, 99% or even 100%.

Again another form of this embodiment relates to a nucleic acid sequence encoding a Mycobacterium avium subspecies paratuberculosis protein or a part of said nucleic acid sequence that encodes an immunogenic fragment of said protein wherein said nucleic acid sequence or said part thereof has at least 85% homology with the nucleic acid sequence of the Mycobacterium avium subspecies paratuberculosis protein gene as depicted in SEQ ID NO: 17.

Preferably, a nucleic acid sequence according to the invention encoding this Mycobacterium avium subspecies paratuberculosis protein or a part of that nucleic acid sequence that encodes an immunogenic fragment of that protein has at least 90%, preferably 93%, more preferably 95% homology with the nucleic acid sequence of the Mycobacterium avium subspecies paratuberculosis protein gene as depicted in SEQ ID NO: 17.

Even more preferred is a homology level of 98%, 99% or even 100%.

A third approach, based upon careful analysis of the proteome of Mycobacterium avium subspecies paratuberculosis has led to the detection of again two novel vaccine components. This approach is based upon identification of immunogenic proteins in 2D-gels. It has the advantage over the other approaches, that proteins not yet found or identified in an expression library can now be unambiguously identified as vaccine components. Details of the methods followed are given in Example 2.

Thus, still another embodiment of the present invention relates to a 60 kD Mycobacterium avium subspecies paratuberculosis protein that has a pI between 5.60 and 6.15.

This protein is visible as a horizontal row of about 5 spots (due to small differences in isoforms representing e.g. different post-translational modifications or artefacts introduced by the preparation of the samples for 2D-gel electrophoresis) in FIGS. 1b and d.

Additionally, another embodiment relates to a 33 kD Mycobacterium avium subspecies paratuberculosis protein that has a pI between 4.20 and 4.75. This protein is visible as a horizontal row of about 3 spots (again due to small differences in isoforms representing e.g. different post-translational modifications or artefacts introduced by the preparation of the samples for 2D-gel electrophoresis) in FIGS. 1a and d.

Since these proteins have now been unambiguously identified, they can be sequenced, e.g. the first 15 N-terminal amino acids can be determined according to standard procedures known in the art. Such N-terminal sequencing is nowadays e.g. commercially and on a routine basis done by companies specialised in protein sequencing. The genes encoding these proteins can then easily be identified using degenerate probes. These techniques are all well-known in the art.

Since the present invention discloses nucleic acid sequences encoding novel Mycobacterium avium subspecies paratuberculosis proteins, it is now for the first time possible to obtain these proteins in sufficient quantities. This can e.g. be done by using expression systems to express the whole or parts of the genes encoding the proteins or immunogenic fragments thereof according to the invention. Therefore, in a more preferred form of the embodiment relating to nucleic acid sequences, the invention relates to DNA fragments comprising a nucleic acid sequence according to the invention. A DNA fragment is a stretch of nucleotides that functions as a carrier for a nucleic acid sequence according to the invention. Such DNA fragments can e.g. be plasmids, into which a nucleic acid sequence according to the invention is cloned. Such DNA fragments are e.g. useful for enhancing the amount of DNA for use as a primer, for DNA-vaccination purposes and for expression of a nucleic acid sequence according to the invention, as described below.

An essential requirement for the expression of nucleic acid sequences is an adequate promoter functionally linked to the nucleic acid sequence, so that the nucleic acid sequence is under the control of the promoter. It is obvious to those skilled in the art that the choice of a promoter extends to any eukaryotic, prokaryotic or viral promoter capable of directing gene transcription in cells used as host cells for protein expression.

Therefore, an even more preferred form of this embodiment relates to a recombinant DNA molecule comprising a DNA fragment and/or a nucleic acid sequence according to the invention wherein the nucleic acid sequence according to the invention is placed under the control of a functionally linked promoter. This can be obtained by means of e.g. standard molecular biology techniques. (Maniatis/Sambrook (Sambrook, J. Molecular cloning: a laboratory manual, 1989. ISBN 0-87969-309-6).

Functionally linked promoters are promoters that are capable of controlling the transcription of the nucleic acid sequences to which they are linked. Such a promoter can be the native promoter of a novel gene according to the invention or another promoter of Mycobacterium avium subspecies paratuberculosts, provided that that promoter is functional in the cell used for expression. It can also be a heterologous promoter. When the host cells are bacteria, useful expression control sequences which may be used include the Trp promoter and operator (Goeddel, et al., Nucl. Acids Res., 8, 4057, 1980); the lac promoter and operator (Chang, et al., Nature, 275, 615, 1978); the outer membrane protein promoter (Nakamura, K. and Inouge, M., EMBO J., 1, 771-775, 1982); the bacteriophage lambda promoters and operators (Remaut, E. et al., Nucl. Acids Res., 11, 4677-4688, 1983); the α-amylase (B. subtilis) promoter and operator, termination sequences and other expression enhancement and control sequences compatible with the selected host cell.

When the host cell is yeast, useful expression control sequences include, e.g., α-mating factor. For insect cells the polyhedrin or p10 promoters of baculoviruses can be used (Smith, G. E. et al., Mol. Cell. Biol. 3, 2156-65, 1983). When the host cell is of vertebrate origin illustrative useful expression control sequences include the (human) cytomegalovirus immediate early promoter (Seed, B. et al., Nature 329, 840-842, 1987; Fynan, E. F. et al., PNAS 90 11478-11482,1993; Ulmer, J. B. et al., Science 259, 1745-1748, 1993), Rous sarcoma virus LTR (RSV, Gorman, C. M. et al., PNAS 79 6777-6781, 1982; Fynan et al., supra; Ulmer et al., supra), the MPSV LTR (Stacey et al., J. Virology 50, 725-732, 1984), SV40 immediate early promoter (Sprague J. et al., J. Virology 45, 773, 1983), the SV-40 promoter (Berman, P. W. et al., Science, 222, 524-527, 1983), the metallothionein promoter (13rinster, R. L. et al., Nature 296. 39-42, 1982), the heat shock promoter (Voellmy et al., Proc. Natl. Acad. Sci. USA, 82, 4949-53, 1985), the major late promoter of Ad2 and the β-actin promoter (Tang et al., Nature 356, 152-154, 1992). The regulatory sequences may also include terminator and poly-adenylation sequences. Amongst the sequences that can be used are the well known bovine growth hormone poly-adenylation sequence, the SV40 poly-adenylation sequence, the human cytomegalovirus (HCMV) terminator and poly-adenylation sequences.

Bacterial, yeast, fungal, insect and vertebrate cell expression systems are very frequently used systems. Such systems are well-known in the art and generally available, e.g. commercially through Clontech Laboratories, Inc. 4030 Fabian Way, Palo Alto, Calif. 94303-4607, USA. Next to these expression systems, parasite-based expression systems are attractive expression systems. Such systems are e.g. described in the French Patent Application with Publication number 2 714 074, and in U.S. NTIS Publication No U.S. Ser. No. 08/043,109 (Hoffman, S. and Rogers, W.: Public. Date 1 Dec. 1993).

A still even more preferred form of this embodiment of the invention relates to Live Recombinant Carriers (LRCs) comprising a nucleic acid sequence encoding an Mycobacterium avium subspecies paratuberculosis protein or an immunogenic fragment thereof according to the invention, a DNA fragment according to the invention or a recombinant DNA molecule according to the invention. These LRCs are micro-organisms or viruses in which additional genetic information, in this case a nucleic acid sequence encoding an Mycobacterium avium subspecies paratuberculosis protein or an immunogenic fragment thereof according to the invention has been cloned. Cattle infected with such LRCs will produce an immunological response not only against the immunogens of the carrier, but also against the immunogenic parts of the protein(s) for which the genetic code is additionally cloned into the LRC, such as e.g. one or more of the novel Mycobacterium avium subspecies paratuberculosis proteins genes according to the invention.

As an example of bacterial LRCs, attenuated Salmonella strains known in the art can very attractively be used.

Also, live recombinant carrier parasites have i.a. been described by Vermeulen, A. N. (Int. Journ. Parasitol. 28: 1121-1130 (1998)).

Furthermore, LRC viruses may be used as a way of transporting the nucleic acid sequence into a target cell. Live recombinant carrier viruses are also called vector viruses. Viruses often used as vectors are Vaccinia viruses (Panicali et al; Proc. Natl. Acad. Sci. USA, 79: 4927 (1982), Herpesviruses (E.P.A 0473210A2), and Retroviruses (Valerio, D. et al; in Baum, S. J., Dicke, K. A., Lotzova, E. and Pluznik, D. H. (EEds.), Experimental Haematology today—1988. Springer Verlag, New York: pp. 92-99 (1989)).

The technique of in vivo homologous recombination, well-known in the art, can be used to introduce a recombinant nucleic acid sequence into the genome of a bacterium, parasite or virus of choice, capable of inducing expression of the inserted nucleic acid sequence according to the invention in the host animal.

Finally another form of this embodiment of the invention relates to a host cell comprising a nucleic acid sequence encoding a protein according to the invention, a DNA fragment comprising such a nucleic acid sequence or a recombinant DNA molecule comprising such a nucleic acid sequence under the control of a functionally linked promoter. This form also relates to a host cell containing a live recombinant carrier comprising a nucleic acid molecule encoding an Mycobacterium avium subspecies paratuberculosis protein or an immunogenic fragment thereof according to the invention.

A host cell may be a cell of bacterial origin, e.g. Escherichia coli, Bacillus subtilis and Lactobacillus species, in combination with bacteria-based plasmids as pBR322, or bacterial expression vectors as the pEX-, pET-, pGEX-series, or with bacteriophages. The host cell may also be of eukaryotic origin, e.g. yeast-cells in combination with yeast-specific vector molecules, or higher eukaryotic cells like insect cells (Luckow et al; Bio-technology 6: 47-55 (1988)) in combination with vectors or recombinant baculoviruses, plant cells in combination with e.g. Ti-plasmid based vectors or plant viral vectors (Barton, K. A. et al; Cell 32: 1033 (1983), mammalian cells like Hela cells, Chinese Hamster Ovary cells (CHO) or Crandell Feline Kidney-cells, also with appropriate vectors or recombinant viruses.

Another embodiment of the invention relates to the novel Mycobacterium avium subspecies paratuberculosis proteins and to immunogenic fragments thereof according to the invention.

The concept of immunogenic fragments will be defined below.

One form of this embodiment relates to a 28 kD Mycobacterium avium subspecies paratuberculosis protein and to immunogenic fragments thereof, wherein the protein or immunogenic fragments have a sequence homology of at least 90%, preferably however 92%, more preferably 94%, 95% or even 96% homology, in that order or preference, to the amino acid sequence as depicted in SEQ ID NO: 2.

Even more preferred is a homology level of 97%, 98%, 99% or even 100% in that order of preference.

The immunogenic fragments of the Mycobacterium avium subspecies paratuberculosis protein as depicted in SEQ ID NO: 2 and in SEQ ID NO: 4, 6, 8, 10, 12, 14, 16 and 18 according to the invention as described below preferably have a length of at least 6, more preferably 7, 8, 9, 10, 12, 15, 20, 30 or even 40 amino acids, in that order of preference.

A still even more preferred form of this embodiment relates to this Mycobacterium avium subspecies paratuberculosis protein and immunogenic fragments of said protein, encoded by a nucleic acid sequence as depicted in SEQ ID NO: 1.

As was mentioned above, bacteria survive inside host cells, but extracellular episodes of infection in the lumen of the gastrointestinal tract do also occur. This implies that both cell-mediated and antibody-mediated immune responses play a role in adequate protection against disease. As is shown in the Examples, tests checking for both T-cell response and B-cell response have been used for the determination of the value of the proteins according to the invention as vaccine components.

Another form of this embodiment relates to a 14 kD Mycobacterium avium subspecies paratuberculosis protein and to immunogenic fragments thereof, wherein the protein or immunogenic fragments have a sequence homology of at least 90%, preferably however 92%, more preferably 94%, 95% or even 96% homology, in that order or preference, to the amino acid sequence as depicted in SEQ ID NO: 4.

Even more preferred is a homology level of 97%, 98%, 99% or even 100% in that order of preference.

A still even more preferred form of this embodiment relates to a 14 kD Mycobacterium avium subspecies paratuberculosis protein and immunogenic fragments of said protein, encoded by a nucleic acid sequence as depicted in SEQ ID NO: 3.

Still another form of this embodiment relates to a 9 kD Mycobacterium avium subspecies paratuberculosis protein and to immunogenic fragments thereof, wherein the protein or immunogenic fragments have a sequence homology of at least 90%, preferably however 92%, more preferably 94%, 95% or even 96% homology, in that order or preference, to the amino acid sequence as depicted in SEQ ID NO: 6.

Even more preferred is a homology level of 97%, 98%, 99% or even 100% in that order of preference.

A still even more preferred form of this embodiment relates to a 9 kD Mycobacterium avium subspecies paratuberculosis protein and immunogenic fragments of said protein, encoded by a nucleic acid sequence as depicted in SEQ ID NO: 5.

Again another form of this embodiment relates to a 47 kD Mycobacterium avium subspecies paratuberculosis protein and to immunogenic fragments thereof, wherein the protein or immunogenic fragments have a sequence homology of at least 90%, preferably however 92%, more preferably 94%, 95% or even 96% homology, in that order or preference, to the amino acid sequence as depicted in SEQ ID NO: 8.

Even more preferred is a homology level of 97%, 98%, 99% or even 100% in that order of preference.

A still even more preferred form of this embodiment relates to a 47 kD Mycobacterium avium subspecies paratuberculosis protein and immunogenic fragments of said protein, encoded by a nucleic acid sequence as depicted in SEQ ID NO: 7.

One other form of this embodiment relates to a Mycobacterium avium subspecies paratuberculosis protein and to immunogenic fragments thereof, wherein the protein or immunogenic fragments have a sequence homology of at least 90%, preferably however 92%, more preferably 94%, 95% or even 96% homology, in that order or preference, to the amino acid sequence as depicted in SEQ ID NO: 10.

Even more preferred is a homology level of 97%, 98%, 99% or even 100% in that order of preference.

A still even more preferred form of this embodiment relates to a Mycobacterium avium subspecies paratuberculosis protein and immunogenic fragments of said protein, encoded by a nucleic acid sequence as depicted in SEQ ID NO: 9.

Again an other form of this embodiment relates to a Mycobacterium avium subspecies paratuberculosis protein and to immunogenic fragments thereof, wherein the protein or immunogenic fragments have a sequence homology of at least 90%, preferably however 92%, more preferably 94%, 95% or even 96% homology, in that order or preference, to the amino acid sequence as depicted in SEQ ID NO: 12.

Even more preferred is a homology level of 97%, 98%, 99% or even 100% in that order of preference.

A still even more preferred form of this embodiment relates to a Mycobacterium avium subspecies paratuberculosis protein and immunogenic fragments of said protein, encoded by a nucleic acid sequence as depicted in SEQ ID NO: 11.

Again another form of this embodiment relates to a Mycobacterium avium subspecies paratuberculosis protein and to immunogenic fragments thereof, wherein the protein or immunogenic fragments have a sequence homology of at least 90%, preferably however 92%, more preferably 94%, 95% or even 96% homology, in that order or preference, to the amino acid sequence as depicted in SEQ ID NO: 14.

Even more preferred is a homology level of 97%, 98%, 99% or even 100% in that order of preference.

A still even more preferred form of this embodiment relates to a Mycobacterium avium subspecies paratuberculosis protein and immunogenic fragments of said protein, encoded by a nucleic acid sequence as depicted in SEQ ID NO: 13.

Again another form of this embodiment relates to a Mycobacterium avium subspecies paratuberculosis protein and to immunogenic fragments thereof, wherein the protein or immunogenic fragments have a sequence homology of at least 90%, preferably however 92%, more preferably 94%, 95% or even 96% homology, in that order or preference, to the amino acid sequence as depicted in SEQ ID NO: 16.

Even more preferred is a homology level of 97%, 98%, 99% or even 100% in that order of preference.

A still even more preferred form of this embodiment relates to a Mycobacterium avium subspecies paratuberculosis protein and immunogenic fragments of said protein, encoded by a nucleic acid sequence as depicted in SEQ ID NO: 15.

Again another form of this embodiment relates to a Mycobacterium avium subspecies paratuberculosis protein and to immunogenic fragments thereof, wherein the protein or immunogenic fragments have a sequence homology of at least 90%, preferably however 92%, more preferably 94%, 95% or even 96% homology, in that order or preference, to the amino acid sequence as depicted in SEQ ID NO: 18.

Even more preferred is a homology level of 97%, 98%, 99% or even 100% in that order of preference.

A still even more preferred form of this embodiment relates to a Mycobacterium avium subspecies paratuberculosis protein and immunogenic fragments of said protein, encoded by a nucleic acid sequence as depicted in SEQ ID NO: 17.

The level of protein homology can be determined with the computer program “BLAST 2 SEQUENCES” by selecting sub-program: “BLASTP”, that can be found at www.ncbi.nhm.nih.gov/blast/bl2seq/bl2.html.

A reference for this program is Tatiana A Tatusova, Thomas L. Madden FEMS Microbiol. Letters 174: 247-250 (1999). Matrix used: “blosum62”. Parameters used are the default parameters:
Open gap: 11. Extension gap: 1. Gap x_dropoff: 50.

It will be understood that, for the particular proteins embraced herein, natural variations can exist between individual Mycobacterium avium subspecies paratuberculosis strains. These variations may be demonstrated by (an) amino acid difference(s) in the overall sequence or by deletions, substitutions, insertions, inversions or additions of (an) amino acid(s) in said sequence. Amino acid substitutions which do not essentially alter biological and immunological activities, have been described, e.g. by Neurath et al in “The Proteins” Academic Press New York (1979). Amino acid replacements between related amino acids or replacements which have occurred frequently in evolution are, inter alia, Ser/Ala, Ser/Gly, Asp/Gly, Asp/Asn, Ile/Val (see Dayhof, M. D., Atlas of protein sequence and structure, Nat. Biomed. Res. Found., Washington D.C., 1978, vol. 5, suppl. 3). Other amino acid substitutions include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, Lipman and Pearson developed a method for rapid and sensitive protein comparison (Science, 227, 1435-1441, 1985) and determining the functional similarity between homologous proteins. Such amino acid substitutions of the exemplary embodiments of this invention, as well as variations having deletions and/or insertions are within the scope of the invention as long as the resulting proteins retain their immune reactivity.

This explains why Mycobacterium avium subspecies paratuberculosis proteins according to the invention, when isolated from different field isolates, may have homology levels of about 70%, while still representing the same protein with the same immunological characteristics.

Those variations in the amino acid sequence of a certain protein according to the invention that still provide a protein capable of inducing an immune response against infection with Mycobacterium avium subspecies paratuberculosis or at least against the clinical manifestations of the infection are considered as “not essentially influencing the immunogenicity”.

When a protein is used for e.g. vaccination purposes or for raising antibodies, it is however not necessary to use the whole protein. It is also possible to use a fragment of that protein that is capable, as such or coupled to a carrier such as e.g. KLH, of inducing an immune response against that protein, a so-called immunogenic fragment. An “immunogenic fragment” is understood to be a fragment of the full-length protein that still has retained its capability to induce an immune response in a vertebrate host, e.g. comprises a B- or T-cell epitope. Shortly, an immunogenic fragment is a fragment that is capable of inducing an immunogenic response against an Mycobacterium avium subspecies paratuberculosis protein according to the invention. At this moment, a variety of techniques is available to easily identify DNA fragments encoding antigenic fragments (determinants). The method described by Geysen et al (Patent Application WO 84/03564, Patent Application WO 86/06487, U.S. Pat. No. 4,833,092, Proc. Natl. Acad. Sci. 81: 3998-4002 (1984), J. 1 mm. Meth. 102, 259-274 (1987), the so-called PEPSCAN method is an easy to perform, quick and well-established method for the detection of epitopes; the immunologically important regions of the protein. The method is used world-wide and as such well-known to man skilled in the art. This (empirical) method is especially suitable for the detection of B-cell and T-cell epitopes. Also, given the sequence of the gene encoding any protein, computer algorithms are able to designate specific protein fragments as the immunologically important epitopes on the basis of their sequential and/or structural agreement with epitopes that are now known. The determination of these regions is based on a combination of the hydrophilicity criteria according to Hopp and Woods (Proc. Natl. Acad. Sci. 78: 38248-3828 (1981)), and the secondary structure aspects according to Chou and Fasman (Advances in Enzymology 47: 45-148 (1987) and U.S. Pat. No. 4,554,101). T-cell epitopes can likewise be predicted from the sequence by computer with the aid of Berzofsky's amphiphilicity criterion (Science 235, 1059-1062 (1987) and US Patent application NTIS U.S. Ser. No. 07/005,885). A condensed overview is found in: Shan Lu on common principles: Tibtech 9: 238-242 (1991), Good et al on Malaria epitopes; Science 235: 1059-1062 (1987), Lu for a review; Vaccine 10: 3-7 (1992), Berzofsky for HIV-epitopes; The FASEB Journal 5:2412-2418 (1991). An immunogenic fragment usually has a minimal length of 6, more commonly 7-8 amino acids, preferably more then 8, such as 9, 10, 12, 15 or even 20 or more amino acids. The nucleic acid sequences encoding such a fragment therefore have a length of at least 18, more commonly 24 and preferably 27, 30, 36, 45 or even 60 nucleic acids.

Therefore, one form of still another embodiment of the invention relates to vaccines for combating Mycobacterium avium subspecies paratuberculosis infection, that comprise at least one Mycobacterium avium subspecies paratuberculosis protein or immunogenic fragments thereof, according to the invention as described above together with a pharmaceutically acceptable carrier.

Still another embodiment of the present invention relates to the Mycobacterium avium subspecies paratuberculosis proteins according to the invention or immunogenic fragments thereof for use in a vaccine.

Again another embodiment of the present invention relates to the use of a nucleic acid sequence, a DNA fragment, a recombinant DNA molecule, a live recombinant carrier, a host cell or a protein or an immunogenic fragment thereof according to the invention for the manufacturing of a vaccine, more specifically a vaccine for combating Mycobacterium avium subspecies paratuberculosis infection.

One way of making a vaccine according to the invention is by growing the bacterium, followed by biochemical purification of an Mycobacterium avium subspecies paratuberculosis protein or immunogenic fragments thereof, from the bacterium or the supernatant. This is however a very time-consuming way of making the vaccine.

It is therefore much more convenient to use the expression products of a gene encoding an Mycobacterium avium subspecies paratuberculosis protein or immunogenic fragments thereof, according to the invention in vaccines. This is possible for the first time now because the nucleic acid sequences of genes encoding 9 novel Mycobacterium avium subspecies paratuberculosis proteins suitable as vaccine components is provided in the present invention.

Vaccines based upon the expression products of these genes can easily be made by admixing the protein according to the invention or immunogenic fragments thereof according to the invention with a pharmaceutically acceptable carrier as described below.

Alternatively, a vaccine according to the invention can comprise live recombinant carriers as described above, capable of expressing the protein according to the invention or immunogenic fragments thereof. Such vaccines, e.g. based upon a Salmonella carrier or a viral carrier e.g. a Herpesvirus vector have the advantage over subunit vaccines that they better mimic the natural way of infection of Mycobacterium avium subspecies paratuberculosis. Moreover, their self-propagation is an advantage since only low amounts of the recombinant carrier are necessary for immunization.

Vaccines can also be based upon host cells as described above, that comprise the protein or immunogenic fragments thereof according to the invention.

The vaccines according to the invention have an additional advantage over e.g. killed whole bacteria vaccines and live attenuated vaccines. Vaccines based upon the whole cell induce antibodies against all antigenic determinants, i.e. all epitopes present on the bacterium. Therefore, the antibody panel raised against such vaccines is comparable to that raised after field infection. As a consequence, it is impossible to tell if an animal has been infected or has been vaccinated. The use of the proteins or immunogenic fragments thereof according to the invention, i.e. subunits of the whole bacterium, as vaccine components has the advantage that vaccinated animals only make antibodies against the administered subunits. A simple comparison of the antibody panel of suspected animals with that of vaccinated and field infected animals will immediately tell if the suspected animal was field-infected or vaccinated. For such tests, to be discussed below in more detail, a simple EUISA test is sufficient. Such vaccines based upon one or more subunits are known as marker vaccines: they are “marked” in the sense that they can be discriminated from field infection. Below, the concept of marker vaccines is discussed in more detail.

Very attractive marker vaccines are vaccines based upon the 9 kD protein or immunogenic fragments thereof, of which the sequence is depicted in SEQ ID NO: 6. The reason for this is the following: there is a relatively high level of cross-reactivity between antibodies raised against Mycobacterium avium subspecies paratuberculosis and Myco bacterium bovis. Myco bacterium bovis is i.a. the cause of bovine tuberculosis. The bacterium is also contagious for other animal species. Moreover, this disease is a zoonotic disease, i.e. it can be transferred to man. The World Health Organization (WHO) estimates that human tuberculosis (TB) incidence and deaths for 1990 to 1999 mounted to 88 million and 30 million, respectively, with most cases in developing countries. Zoonotic TB (caused by Mycobacterium bovis) is present in animals in most developing countries where surveillance and control activities are often inadequate or unavailable; therefore, many epidemiological and public health aspects of infection remain largely unknown.

The fact that M. bovis is contagious for other mammals is one of the reasons to attempt to eradicate M bovis. One of the measures necessary to obtain this goal, is to eradicate cattle that is found positive for M. bovis in diagnostic tests such as the bovine PPD DTH test. And as mentioned above, due to cross-reactivity of sera against M. bovis and Mycobacterium avium subspecies paratuberculosis animals found positive in this test are eradicated, regardless the cause of infection.

Therefore, a diagnostic test that can clearly discriminate between vaccination with a subunit vaccine according to the invention and field infection with either Mycobacterium bovis or Mycobacterium avium subspecies paratuberculosis would be a very valuable tool.

It is one of the merits of the present invention that Mycobacterium avium subspecies paratuberculosis proteins were found that do not show cross reactivity with Mycobacterium bovis in a PPD test. The protein of which the amino acid sequence is given in SEQ ID NO: 6 belongs to these proteins.

Therefore, a preferred form of this embodiment relates to vaccines comprising a protein as depicted in SEQ ID NO: 6 or an immunogenic fragment thereof.

If a vaccine is wanted that does not necessarily have as a property that it can be used for marker purposes as described above, the addition of other proteins according to the invention or the 65 kD or 70 kD heat-shock protein, or immunogenic fragments thereof would be beneficial. Such combination vaccines enhance the efficacy of the vaccine, when compared to single vaccines. Therefore, in a more preferred form, a vaccine according to the invention comprises both the 9 kD protein one or more of the other proteins according to the invention or one of the heat shock proteins or immunogenic fragments thereof.

All vaccines described above contribute to active vaccination, ie. they trigger the host's defence system.

Alternatively, antibodies can be raised in e.g. rabbits or can be obtained from antibody-producing cell lines as described below. Such antibodies can then be administered to the mammal to be vaccinated/protected. This method of vaccination, passive vaccination, is the vaccination of choice when a mammal is already infected, and there is no time to allow the natural immune response to be triggered. It is also the preferred method for vaccinating mammals that are prone to sudden high infection pressure and to immune compromised individuals. The administered antibodies against the protein according to the invention or immunogenic fragments thereof can in these cases interfere with Mycobacterium avium subspecies paratuberculosis. This approach has the advantage that it decreases or stops Mycobacterium avium subspecies paratuberculosis development. Therefore, one other form of this embodiment of the invention relates to a vaccine for combating Mycobacterium avium subspecies paratuberculosis infection that comprises antibodies against an Mycobacterium avium subspecies paratuberculosis protein according to the invention or an immunogenic fragment of that protein, and a pharmaceutically acceptable carrier.

Still another embodiment of this invention relates to antibodies against an Mycobacterium avium subspecies paratuberculosis protein according to the invention or an immunogenic fragment of that protein.

Methods for large-scale production of antibodies according to the invention are also known in the art. Such methods rely on the cloning of (fragments of) the genetic information encoding the protein according to the invention in a filamentous phage for phage display. Such techniques are described i.a. at the “Antibody Engineering Page” under “filamentous phage display” at http://axint1.imt.uni-marburg.de/˜rek/aeophage.html. and in review papers by Cortese, R. et al., (1994) in Trends Biotechn. 12: 262-267, by Clackson, T. & Wells, J. A. (1994) in Trends Biotechn. 12: 173-183, by Marks, J. D. et al., (1992) in J. Biol. Chem. 267: 16007-16010, by Winter, G. et al., (1994) in Annu. Rev. Immunol. 12: 433-455, and by Little, M. et al., (1994) Biotechn. Adv. 12: 539-555. The phages are subsequently used to screen camelid expression libraries expressing camelid heavy chain antibodies. (Muyldermans, S. and Lauwereys, M., Journ. Molec. Recogn. 12: 131-140 (1999) and Ghahroudi, M. A. et al., FEBS Letters 414: 512-526 (1997)). Cells from the library that express the desired antibodies can be replicated and subsequently be used for large scale expression of antibodies.

Still another embodiment relates to a method for the preparation of a vaccine according to the invention that comprises the admixing of antibodies according to the invention and a pharmaceutically acceptable carrier.

An alternative and efficient way of vaccination is direct vaccination with DNA encoding the relevant antigen. Direct vaccination with DNA encoding proteins has been successful for many different proteins. (As reviewed in e.g. Donnelly et al., The Immunologist 2: 20-26 (1993)). More specifically, protection against Mycobacterium avium by DNA vaccination has been described by Velaz-Faircloth, M. et al., (Infect. & Immun. 67: 4243-4250 (1999)).

This way of vaccination is very attractive for the vaccination of cattle against Mycobacterium avium subspecies paratuberculosis infection. Therefore, still other forms of this embodiment of the invention relate to vaccines comprising nucleic acid sequences encoding a protein according to the invention or immunogenic fragments thereof, vaccines comprising DNA fragments that comprise such nucleic acid sequences or vaccines comprising recombinant DNA molecules according to the invention, and a pharmaceutically acceptable carrier.

Preferably, nucleic acid sequences according to the invention encoding the 9 kD protein or an immunogenic fragment thereof and described in SEQ ID NO: 5 are used for vaccination, for reasons given above.

More preferably, such sequences are combined with sequences encoding another protein or an immunogenic fragment thereof according to the invention as described above.

Examples of DNA plasmids that are suitable for use in a DNA vaccine according to the invention are conventional cloning or expression plasmids for bacterial, eukaryotic and yeast host cells, many of said plasmids being commercially available. Well-known examples of such plasmids are pBR322 and pcDNA3 (Invitrogen). The DNA fragments or recombinant DNA molecules according to the invention should be able to induce protein expression of the nucleotide sequences. The DNA fragments or recombinant DNA molecules may comprise one or more nucleotide sequences according to the invention. In addition, the DNA fragments or recombinant DNA molecules may comprise other nucleotide sequences such as immune-stimulating oligonucleotides having unmethylated CpG di-nucleotides, or nucleotide sequences that code for other antigenic proteins or adjuvating cytokines.

The nucleotide sequence according to the present invention or the DNA plasmid comprising a nucleotide sequence according to the present invention, preferably operably linked to a transcriptional regulatory sequence, to be used in the vaccine according to the invention can be naked or can be packaged in a delivery system.

Suitable delivery systems are lipid vesicles, iscos, dendromers, niosomes, microparticles, especially chitosan-based microparticles, polysaccharide matrices and the like, (see further below) all well-known in the art. Also very suitable as delivery system are attenuated live bacteria such as Salmonella species, and attenuated live viruses such as Herpesvirus vectors, as mentioned above.

Still other forms of this embodiment relate to vaccines comprising recombinant DNA molecules according to the invention.

DNA vaccines can e.g. easily be administered through intradermal application such as by using a needle-less injector. This way of administration delivers the DNA directly into the cells of the animal to be vaccinated. Amounts of DNA in the range between 10 pg and 1000 μg provide good results. Especially if the DNA is self-replicating, minor amounts will suffice. Preferably, amounts in the microgram range between 1 and 100 μg are used.

In a further embodiment, the vaccine according to the present invention additionally comprises one or more antigens derived from cattle pathogenic organisms and viruses, antibodies against those antigens or genetic information encoding such antigens and/or a pharmaceutical component such as an antibiotic. Of course, such antigens, antibodies against such antigens, or genetic information can be of Mycobacterium avium subspecies paratuberculosis origin, such as e.g. another Mycobacterium avium subspecies paratuberculosis antigen. It can also be an antigen, antibodies or genetic information selected from another cow pathogenic organism or virus. Such organisms and viruses are preferably selected from the group of Bovine Herpesvirus, bovine Viral Diarrhoea virus, Parainfluenza type 3 virus, Bovine Paramyxovirus, Foot and Mouth Disease virus, Pasteurella haemolytica, Bovine Respiratory Syncytial Virus, Theileria sp., Babesia sp., Trypanosoma species, Anaplasma sp., Neospora caninum, Staphylococcus aureus, Streptococcus agalactiae, Mycoplasma, E. coli, Enterobacter, Klebsiella, Citrobacter and Streptococcus dysgalactiae.

As mentioned earlier, vaccines based upon one or more of the Mycobacterium avium subspecies paratuberculosis proteins according to the invention are also very suitable as marker vaccines. A marker vaccine is a vaccine that allows to discriminate between vaccinated and field-infected mammals e.g. on the basis of a characteristic antibody panel, different from the antibody panel induced by wild type infection. A different antibody panel is induced e.g. when an immunogenic protein present on a wild type Mycobacterium avium subspecies paratuberculosis is not present in a vaccine: the host will then not make antibodies against that protein after vaccination. Thus, a vaccine based upon any of the Mycobacterium avium subspecies paratuberculosis proteins according to the invention would only induce antibodies against that specific protein, whereas a vaccine based upon a live wild-type, live attenuated or inactivated whole Mycobacterium avium subspecies paratuberculosis would induce antibodies against all or most of the bacterial proteins.

A simple ELISA test, having wells comprising any other Mycobacterium avium subspecies paratuberculosis protein except for a Mycobacterium avium subspecies paratuberculosis protein according to the present invention and wells comprising only one or more purified Mycobacterium avium subspecies paratuberculosis proteins according to the invention suffices to test serum from cows and to tell if the cows are either vaccinated with the protein vaccine according to the invention or suffered from Mycobacterium avium subspecies paratuberculosis field infection.

All vaccines according to the present invention comprise a pharmaceutically acceptable carrier. A pharmaceutically acceptable carrier can be e.g. sterile water or a sterile physiological salt solution. In a more complex form the carrier can e.g. be a buffer.

Methods for the preparation of a vaccine comprise the admixing of a protein or an immunogenic fragment thereof, according to the invention and/or antibodies against that protein or an immunogenic fragment thereof, and/or a nucleic acid sequence and/or a DNA fragment, a recombinant DNA molecule, a live recombinant carrier or host cell according to the invention, and a pharmaceutically acceptable carrier.

Vaccines according to the present invention may in a preferred presentation also contain an immunostimulatory substance, a so-called adjuvant. Adjuvants in general comprise substances that boost the immune response of the host in a non-specific manner. A number of different adjuvants are known in the art. Examples of adjuvants frequently used in cow vaccines are muramyldipeptides, lipopolysaccharides, several glucans and glycans and Carbopol^{(R)} (a homaopolymer). The vaccine may also comprise a so-called “vehicle”. A vehicle is a compound to which the protein adheres, without being covalently bound to it. Such vehicles are i.a. bio-microcapsules, micro-alginates, liposomes and macrosols, all known in the art. Microparticles, more specifically those based upon chitosan, especially for use in oral vaccination are very suitable as vaccine vehicles.

A special form of such a vehicle, in which the antigen is partially embedded in the vehicle, is the so-called ISCOM (EP 109.942, EP 180.564, EP 242.380) In addition, the vaccine may comprise one or more suitable surface-active compounds or emulsifiers, e.g. Span or Tween.

Antigens will preferably be combined with adjuvants that are readily available and that are registered for use in domestic animals and/or humans, e.g. aluminium hydroxide, a Th2-like modulating adjuvant.

Addition of CpG oligonucleotide sequences inside or outside the plasmid is also preferred for improving protection.

Often, the vaccine is mixed with stabilisers, e.g. to protect degradation-prone proteins from being degraded, to enhance the shelf-life of the vaccine, or to improve freeze-drying efficiency. Useful stabilisers are i.a. SPGA (Bovarnik et al; J. Bacteriology 59: 509 (1950)), carbohydrates e.g. sorbitol, mannitol, trehalose, starch, sucrose, dextran or glucose, proteins such as albumin or casein or degradation products thereof, and buffers, such as alkali metal phosphates. In addition, the vaccine may be suspended in a physiologically acceptable diluent. It goes without saying, that other ways of adjuvating, adding vehicle compounds or diluents, emulsifying or stabilising a protein are also embodied in the present invention.

Vaccines according to the invention that are based upon the protein according to the invention or immunogenic fragments thereof can very suitably be administered in amounts ranging between 1 and 100 micrograms of protein per animal, although smaller doses can in principle be used. A dose exceeding 100 micrograms will, although immunologically very suitable, be less attractive for commercial reasons. Vaccines based upon live attenuated recombinant carriers, such as the LRC-viruses, parasites and bacteria described above can be administered in much lower doses, because they multiply themselves during the infection. Therefore, very suitable amounts would range between 10³ and 10⁹ CFU/PFU for both bacteria and viruses.

Vaccines according to the invention can be administered e.g. intradermally, subcutaneously, intramuscularly, intraperitoneally, intravenously, or at mucosal surfaces such as orally or intranasally.

For efficient protection against disease, a quick and correct diagnosis of Mycobacterium avium subspecies paratuberculosis infection is important. Therefore it is another objective of this invention to provide diagnostic tools suitable for the detection of Mycobacterium avium subspecies paratuberculosis infection.

The nucleic acid sequences, the proteins and the antibodies according to the invention are also suitable for use in diagnostics.

Therefore, another embodiment of the invention relates to nucleic acid sequences, proteins and antibodies according to the invention for use in diagnostics.

The nucleic acid sequences or fragments thereof according to the invention can be used to detect the presence of Mycobacterium avium subspecies paratuberculosis in cows. A sample taken from a mammal infected with Mycobacterium avium subspecies paratuberculosis will comprise nucleic acid material derived from said bacterium, including nucleic acid sequences encoding for the protein according to the invention. These nucleic acid sequences will hybridize with a nucleic acid sequence according to the invention. Suitable methods for the detection of nucleic acid sequences that are reactive with the nucleic acid sequences of the present invention include hybridization techniques including but not limited to PCR techniques and NASBA techniques. Thus the nucleic acid sequences according to the invention can be used to prepare probes and primers for use in PCR and or NASBA techniques.

A diagnostic test kit for the detection of Mycobacterium avium subspecies paratuberculosis may e.g. comprise tools to enable the reaction of Mycobacterium avium subspecies paratuberculosis nucleic acid isolated from the cows to be tested with these tools. Such tools are e.g. specific probes or (PCR-) primers, also referred to as primer fragments, based upon the nucleic acid sequences according to the invention. If genetic material of Mycobacterium avium subspecies paratuberculosis is present in the animal this will e.g. specifically bind to specific PCR-primers and, e.g. after cDNA synthesis, will subsequently become amplified in PCR-reaction. The PCR-reaction product can then easily be detected in DNA gel electrophoresis. Standard PCR-textbooks give methods for determining the length of the primers for selective PCR-reactions with Mycobacterium avium subspecies paratuberculosis DNA Primer fragments with a nucleotide sequence of at least 12 nucleotides are frequently used, but primers of more than 15, more preferably 18 nucleotides are somewhat more selective. Especially primers with a length of at least 20, preferably at least 30 nucleotides are very generally applicable. PCR-techniques are extensively described in Dieffenbach & Dreksler; PCR primers, a laboratory manual. ISBN 0-87969-447-5 (1995).

Nucleic acid sequences according to the invention or primers of those nucleic acid sequences having a length of at least 12, preferably 15, more preferably 18, even more preferably 20, 22, 25, 30, 35 or 40 nucleotides in that order of preference, wherein the nucleic acid sequences or parts thereof have at least 70% homology with the nucleic acid sequence as depicted in SEQ ID NO: 1, 3, 5, 7, 9, 11, 13, 15 or 17 are therefore also part of the invention. Primers are understood to have a length of at least 12 nucleotides and a homology of at least 70%, more preferably 80%, 85%, 90%, 95%, 98%, 99% or even 100%, in that order of preference, with the nucleic acid sequence as depicted in SEQ ID NO: 1, 3, 5, 7, 9, 11, 13, 15 or 17. Such nucleic acid sequences can be used as primer fragments in PCR-reactions in order to enhance the amount of DNA that they encode or in hybridization reactions. This allows the quick amplification or detection on blots of specific nucleotide sequences for use as a diagnostic tool for e.g. the detection of Mycobacterium avium subspecies paratuberculosis as indicated above.

Another test on genetic material is based upon Mycobacterium avium subspecies paratuberculosis material obtained from e.g. a swab, followed by classical DNA purification followed by classical hybridization with radioactively or colour-labelled primer fragments. Colour-labelled and radioactively labelled fragments are generally called detection means. Both PCR-reactions and hybridization reactions are well-known in the art and are i.a. described in Maniatis/Sambrook (Sambrook, J. et al. Molecular cloning: a laboratory manual. ISBN 0-87969-309-6).

Thus, one embodiment of the invention relates to a diagnostic test kit for the detection of Mycobacterium avium subspecies paratuberculosis nucleic acid sequences. Such a test comprises a nucleic acid sequence according to the invention or a primer fragment thereof.

A diagnostic test kit based upon the detection of antigenic material of the specific Mycobacterium avium subspecies paratuberculosis proteins according to the invention and therefore suitable for the detection of Mycobacterium avium subspecies paratuberculosis infection may i.a. comprise a standard ELISA test. In one example of such a test the walls of the wells of an ELISA plate are coated with antibodies directed against any of the proteins according to the invention. After incubation with the material to be tested, labelled anti-Mycobacterium avium subspecies paratuberculosis antibodies are added to the wells. A colour reaction then reveals the presence of antigenic material from Mycobacterium avium subspecies paratuberculosis.

Therefore, still another embodiment of the present invention relates to diagnostic test kits for the detection of antigenic material of Mycobacterium avium subspecies paratuberculosis. Such test kits comprise antibodies against a protein according to the invention or a fragment thereof according to the invention.

A diagnostic test kit based upon the detection in serum of antibodies against a protein of Mycobacterium avium subspecies paratuberculosis according to the invention and therefore suitable for the detection of Mycobacterium avium subspecies paratuberculosis infection may i.a. comprise a standard ELISA test. In such a test the walls of the wells of an ELISA plate can e.g. be coated with an Mycobacterium avium subspecies paratuberculosis protein according to the invention. After incubation with the material to be tested, labelled anti-bodies against that protein are added to the wells. A lack of colour reaction then reveals the presence of antibodies against Mycobacterium avium subspecies paratuberculosis.

Therefore, still another embodiment of the present invention relates to diagnostic test kits for the detection of antibodies against Mycobacterium avium subspecies paratuberculosis. Such test kits comprise an Mycobacterium avium subspecies paratuberculosis protein according to the invention or a fragment thereof according to the invention.

The design of the immunoassay may vary. For example, the immunoassay may be based upon competition or direct reaction. Furthermore, protocols may use solid supports or may use cellular material. The detection of the antibody-antigen complex may involve the use of labelled antibodies; the labels may be, for example, enzymes, fluorescent-, chemoluminescent-, radio-active- or dye molecules. Suitable methods for the detection of antibodies reactive with a protein according to the present invention in the sample include the enzyme-linked immunosorbent assay (ELISA), immunofluorescense test (IF) and Western blot analysis.

The proteins or immunogenic fragments thereof according to the invention e.g. expressed as indicated above can be used to produce antibodies, which may be polyclonal, monospecific or monoclonal (or derivatives thereof). If polyclonal antibodies are desired, techniques for producing and processing polyclonal sera are well-known in the art (e.g. Mayer and Walter, eds. Immunochemical Methods in Cell and Molecular Biology, Academic Press, London, 1987).

Monoclonal antibodies, reactive against the protein according to the invention or an immunogenic fragment thereof according to the present invention, can be prepared by immunizing inbred mice by techniques also known in the art (Kohler and Milstein, Nature, 256, 495-497, 1975).

It was found, that Mycobacterium avium subspecies avium has an extremely high homology with Mycobacterium avium subspecies paratuberculosis. Mycobacterium avium subspecies avium is found, with a significantly increasing incidence, in pigs but also more and more frequently in humans, especially in immune deficient humans such as HIV-positives.

The proteins according to the invention as mentioned above can therefore equally well be used for vaccination purposes against Mycobacterium avium subspecies avium in both pigs and humans, but also for diagnostic purposes in pigs and humans.

### EXAMPLES

### Example 1

Screening of expression library. In order to identify and characterize antigens in M. avium subsp. paratuberculosis for use in diagnostics, therapeutics and vaccines, a genomnic expression library was constructed using the lambda TripleEx expression vector according to the Clontech manual (pT3003-1) and Stratagene Gigapac III Gold Packaging manual. Briefly, bacterial genomic DNA isolated from M. avium subsp. paratuberculosis strain 316F was partially digested with Tsp509I and fragments of average size of 2.5 kilobase pairs, obtained by sucrose gradient centrifugation, were ligated to EcoRI-digested, dephosphorylated lambda TriplEx arms. The packaging reaction was carried out using Gigapack III Gold Packaging Extract and host strain E. coli XL1Blue (Clontech (S0924)). After plating of the library, immunoscreening of approximately 10⁶ phage plaque's was carried out with 1) a positive bovine serum (designated as 3869) and 2) specific anti-Mycobacterium avium subspecies paratuberculosis monoclonal antibodies. This resulted in the selection of 125 positive lambda TriplEx recombinants. Hundred and seventeen of these 125 positive phage recombinants were successfully converted to plasmid (pTriplEx) recombinants using the protocol described in the Clontech manual (PT3003-1).

DNA sequencing of these 117 pTriplEx recombinants allowed them to be categorized into different antigen groups with each group expressing a different antigenic protein or fragment thereof SEQ 2, 4 and 6 were found in recombinants isolated with serum 3869, SEQ 8 in recombinants isolated with monoclonal antibodies to FabG4, and SEQ 10,12,14,16 and 18 in recombinants isolated with 5 respective monoclonal antibodies (13.67.1A; 10.65.3B; 13.67.2A; 10.32.3B; and 10.66.4B) directed to 5 antigenic molecules of M. avium subsp paratuberculosis. Blast searches against various data bases containing mycobacterial genomic information allowed further characterization of a number of the antigenic polypeptides and their encoding genes. Except for hsp65 and hsp70 heat shock protein antigens found and described in SEQ. ID. No: 19 and 21, none of the here provided antigenic fragments have so far been identified as a for M. avium subsp. paratuberculosis relevant antigen or figure among the already known antigens discussed above for M. avium subsp. paratuberculosis.

### Example 2

Proteomics Approach for Identification of Relevant Proteins.

a) Sample preparation. Cells were harvested from a culture of M. avium sups. paratuberculosis 316F in Watson and Reed culture medium by centrifugation. The cell pellet was washed once with PBS (10 g pellet/40 ml PBS), and stored at −80° C. in 5 ml portions. After thawing, each sample was washed twice with 100 ml cold PBS (4° C.), and suspended in 5 ml cold PBS. Proteinase inhibitors were added (pepstatin 12.5 ug; leupeptin 25 ug, Pefabloc™SC 125 ug; aprotinine 5 ug); and the suspension was sonicated using a Branson sonifier 250 for 10′ at 100% output with 50% interval, on ice. Subsequently Ureum (9M), DTT (70 mM), and Triton X-100 (2%) was added, and the solution was kept at RT for 30′ with occasional shaking. The suspension was subsequently centrifuged for 15′ at 5,000 g at 16° C., and again for 30′ at 100,000 g at 16° C. The resulting samples were subsequently treated with the PlusOne 2-D clean up kit (Amersham Biosciences) to remove trace amount of salts, polysaccharides, nucleic acids, and lipids according to the protocol provided by the manufacturer. The protein concentration of the sample was determined using RC DC Protein Assay (Bio-Rad Laboratories), and the samples were stored at −80° C. until 2D-PAGE. Typically, approximately 100 ug of protein sample was used in 2D-PAGE when followed by silver staining or immune blotting, and up to 1500 ug of protein sample was used when 2D-PAGE was followed by Coomasie Brilliant Blue staining.

b) 2D-PAGE. Iso-electric focusing (EF) was carried out using the Ettan IPGphor Iso-electric Focusing system (Amersham Biosciences) with rehydration of IPGphor strips and IEF using ceramic strip holders according to the protocols provided by the manufacturer. Typically, rehydration and protein loading of 24 cm strips was carried out by adding 450 ul of the protein sample in rehydratation buffer containing 1.4 mg DTT and 0.5% ul IPG-buffer followed by incubation O/N at 20° C. Subsequently, IEF was carried out according to instructions provided by the manufacturers: After IEF, strips can be stored at −20° C. until second-dimension PAGE. For second dimension PAGE, strips were equilibrated by shaking in 14 ml equilibration buffer containing 140 mg DTT for 15′ at RT, and subsequently by shaking in 15 ml equilibration buffer containing 350 mg Iodoacetamide for 15′ at RT. Subsequently, strips were briefly dipped in cathode buffer and applied for second dimension PAGE. Electrophoresis was carried out in 12.6% Ettan Dalt II gels (26×20 cm; 1 mm thickness) using a Ettan Dalttwelve large format vertical system according to the instructions provided by the manufacturers (Amersham Biosciences). For silver staining, gels were fixed in a 40% ethanol, 10% acetic acid solution and stained using plus one silver stain kit (Amersham Biosciences). For Coomasie Brilliant Blue staining, gels were stained using PhastGel Blue R (Amersham Biosciences) according to the instructions provided by the manufacturers. For immune blotting, proteins were transferred to nitrocellulose (Nitrocellulose BA85; Schleicher and Schuell) using the Trans-Blot SD Semi Dry Electrophoretic Transfer Cell (Bio-Rad Laboratories) according to the instructions provided by the manufacturers. Recognition of protein spots by monoclonal or polyclonal antibodies was carried out using a standard protocol using PBS buffer with 5% skimmed milk, rabbit anti-mouse or anti-cow antibodies conjugated to horse radish peroxidase, and peroxide, TMB and DONS as substrate.

Results: as follows from FIG. 1, this approach led to the detection of two immunologically highly relevant proteins.

The one protein is a 60 kD Mycobacterium avium subspecies paratuberculosis protein having a p1 between 5.60 and 6.15. This protein is visible as a horizontal row of about 5 spots in FIG. 1b and d.

The other protein is a 33 kD Mycobacterium avium subspecies paratuberculosis protein having a pI between 4.20 and 4.75. This protein is visible as a horizontal row of about 3 spots in FIGS. 1a and d.

### Example 3

Recognition of the 14 kD Protein, the 9 kD protein and Hsp70 and Hsp65 by T-Cells from Vaccinated and Infected Goats.

Stimulation of T-cells in peripheral blood samples from goats experimentally infected with M. avium subsp. paratuberculosis was detected using a bovine interferon gamma test (BOVIGAM; CSL laboratories Parkville, Victoria, Australia) according to protocol provided by the manufacturers. The following antigens were added to 1.5 ml of whole blood recombinant purified 14 kD protein, 9 kD protein, Hsp70 and Hsp65 (0.5 and 5 ug), and three PPDs (3 ug) derived from M. bouis strain AN5 (produced by ID-Lelystad, The Netherlands), derived from M. avium subsp. avium strain D4 produced by ED-Lelystad, The Netherlands), and derived from M. avium subsp. paratuberculosis strains 3+5/C (produced by ID-Lelystad, The Netherlands), respectively. Each of these antigens were tested at three consecutive times using bi-weekly samples from 9 goats that approximately two years earlier were 12 times orally infected with 1 ml (OD660=0.059) of M. avium subsp. paratuberculosis strain DSU no. 405650 (over a 4 week period: each week on Mondays, Wednesdays and Fridays). Five of these animals (188-193) were vaccinated with 0.5 ml of an experimental killed vaccine based on attenuated M. avium subsp. paratuberculosis strain 316F (produced by D-Lelystad, The Netherlands) 4 weeks before infection. Absorbance values≧0.1 (when corrected for background values) and ≧2 times background values were regarded to show increased production of interferon-gamma due to the presence of antigen.

Results: all recombinant antigens tested induced increased interferon-gamma production in at least one of the animals. This shows that they all play a role in T-cell-mediated immune response. A typical experiment is shown in Table 1. Five out of 9 animals showed an increased response to the 9 kD antigen (56%), three out of nine animals (33%) to the 14 kD, 8 out of 9 (90%) to hsp65, and three out of nine (33%) to hsp70.

TABLE 1

| Exp 5. 040901 | 188 | 189 | 190 | 191 | 193 | 194 | 195 | 196 | 198 |
|---|---|---|---|---|---|---|---|---|---|
| AG | | | | | | | | | |
| Bovis PPD (3 ug) | 1.778 | 1.089 | 0.426 | 1.167 | 0.475 | 0.174 | 0.162 | 0.054 | 0.701 |
| Avium PPD (3 ug) | 3.484 | 1.795 | 1.348 | 3.475 | 3.114 | 0.731 | 0.655 | 0.099 | 2.466 |
| Paratb PPD (3 ug) | 3.501 | 2.664 | 2.187 | >4 | 3.319 | 1.919 | 1.339 | 0.254 | 3.078 |
| 14 kD 0.5 ug | 0.022 | −0.017 | 0.102 | −0.023 | 0.022 | 0.103 | 0.004 | −0.008 | 0.392 |
| 14 kD 5 ug | 0.018 | 0.023 | 0.175 | −0.017 | 0.011 | 0.151 | 0.131 | −0.002 | 0.333 |
| 9 kD 0.5 ug | 0.103 | 0.424 | 0.213 | 0.006 | 0.149 | 0.409 | 0.025 | 0.036 | 0.022 |
| 9 kD 5 ug | 0.139 | 1.169 | 0.262 | 0.009 | 0.065 | 1.712 | 0.243 | 0.045 | 0.107 |
| 47 kD 0.5 ug | −0.022 | −0.020 | −0.012 | −0.040 | −0.002 | −0.007 | −0.006 | −0.007 | −0.002 |
| 47 kD 5 ug | −0.006 | 0.001 | −0.007 | −0.041 | −0.010 | 0.019 | 0.009 | −0.005 | −0.010 |
| Hsp70 0.5 ug | 0.092 | −0.012 | 0.143 | −0.006 | −0.011 | 0.286 | 0.007 | −0.010 | 0.070 |
| Hsp70 5 ug | 0.174 | 0.032 | 0.220 | 0.023 | 0.001 | 0.969 | 0.094 | 0.001 | 0.187 |
| Hsp65 0.5 ug | 0.633 | 0.945 | 0.390 | 0.105 | 0.111 | 0.247 | 0.456 | −0.001 | 0.121 |
| Hsp65 5 ug | 1.069 | 0.949 | 0.925 | 0.188 | 0.357 | 1.086 | 0.599 | 0.049 | 0.213 |

### Example 4

Immunisation of Calves with the 14 kD, 9 kD, 47 kD, 70 kD and 65 kD proteins, detection of specific antibody responses, and detection DTH-reactivity.

a) Immunisation.

In order to evaluate the immunogenicity of the 14 kD, 9 kD, 47 kD, 70 kD and 65 kD proteins and the ability to induce a (cross-reactive) DTH response to PPD derived from M. bovis, calves were immunised as follows: 1) with killed whole cell vaccine based on attenuated M. avium subspecies paratuberculosis 316F (produced by ID-Lelystad, The Netherlands; 4 animals) 2) purified recombinant 14 kD protein in a W/O adjuvant (4 animals) 3) purified recombinant 9 kD protein in a W/O adjuvant (4 animals) 4) purified recombinant 47 kD protein in a W/O adjuvant (4 animals) 5) purified recombinant Hsp70 in a W/O adjuvant (2 animals) 6) purified recombinant Hsp65 in a W/O adjuvant (2 animals) 7) a W/O adjuvant alone (3 animals). Prime and boost immunisations of the following amounts of antigen were given at day 0 and day 127:

| Antigen | prime (ug) | booster (ug) |
|---|---|---|
| 14 kD | 207 | 259 |
| 9 kD | 156 | 236 |
| 47 kD | 273 | 305 |
| 70 kD | 348 | 342 |
| 65 kD | 681 | 491 |

Immunisations with the experimental vaccine were given at day 0 (1 ml) and 127 (0.5 ml). Serum samples were taken at day 52 and day 178. DTH tests were done at day-56 (to establish DTH status of animals before immunisation), and at day 52 and 178.

b) Antibody Detection in Serum.

Immunogenicity of the antigens and their presence in PPD was established by detection of total IgG antibodies in serum samples from one representative animal from each immunisation group using a standard SDS-PAGE and immunoblotting protocol where lanes were loaded with 2.5 ug of purified recombinant 14 kD, 9 kD, 47 kD, 65 kD and 70 kD proteins, and 2.5 ug of various extracts (whole cell sonicate and PPD derived from M. bovis strain AN5; whole cell sonicate from M. avium paratuberculosis strain B854; PPD derived from M. avium paratuberculosis strains 3+5/C).

Results: Antibodies in all representative sera detected the corresponding recombinant 14, 9, 47, 65, and 70 kD proteins (FIG. 2 panels A-E, lanes 1); Antibodies to recombinant 14 kD protein failed to recognise a corresponding antigen in M. bovis and M. avium paratuberculosis whole cell sonicates and PPDs (FIG. 2 panel A, lanes 2-4); Antibodies to recombinant 9 kD protein recognised a corresponding antigen in M. bovis and M. avium paratuberculosis whole cell sonicates and M. avium paratuberculosis PPD, but did not recognise a corresponding protein in M. bovis PPD (FIG. 2 panel B, lanes 2-4); Antibodies to recombinant 47 kD protein recognised a (weak) corresponding antigen in M. bovis and M. avium paratuberculosis whole cell sonicates, but failed to recognise a corresponding band in PPDs (FIG. 2 panel C, lanes 2-4); Antibodies to recombinant 65 and 70 kD proteins recognised a corresponding antigen in M. bovis and M. avium paratuberculosis whole cell sonicates and PPDs (FIG. 2 panels D and E, lanes 2-4).

Additionally, immunogenicity of the antigens was established by detection of total IgG antibodies in serum samples using a standard ELISA protocol where wells were coated with 5 ug of various extracts (whole cell sonicates, KCL extracts, secreted proteins) of various M. avium paratuberculosis strains (B854, 5255, 316F, 3+5/C, Teps) or M. bouis strain AN5, and with 5 ug of recombinant purified 14 kD, 9 kD, 47 kD, 70 kD and 65 kD proteins. Arbitrarily, titers above 1/80 at OD=1.0 were regarded as indicative for a positive response.

Results: strong specific antibody responses were detected to the 14 kD, 47 kD, 70 kD and 65 kD proteins after primary and boost immunisation (titers>640; Table 2 and 3). Specific antibody titers to the 9 kD protein were detected after a boost immunisation (titers 160; Table 2 and 3).

TABLE 2

| | | Serum titers at OD = 1.0 (day 52) | | | | | | | | | | |
|---|---|---|---|---|---|---|---|---|---|---|---|---|
| | | B854 | | | | | | | | | | |
| | | Form | 5255 | 3 + 5/C | 316F | Teps | AN5 | | | | | |
| | | Cells | USO | John. | Excr. | KCL | KCL | 14 kD | 9 kD | 47 kD | Hsp70 | Hsp65 |
| Paratb | 840 | >640 | >640 | >640 | >640 | >640 | >640 | <5 | <5 | 5 | 40 | <5 |
| | 6431 | >640 | >640 | >640 | >640 | >640 | >640 | <5 | <5 | <5 | 40 | 10 |
| | 7029 | >640 | >640 | >640 | >640 | >640 | >640 | <5 | <5 | <5 | 20 | 5 |
| | 9293 | >640 | >640 | >640 | >640 | >640 | >640 | <5 | 5 | <5 | 40 | 10 |
| 14 kD | 5955 | 10 | 20 | <5 | 10 | 20 | 40 | 320 | <5 | <5 | 5 | 20 |
| | 7002 | <5 | 5 | <5 | <5 | 10 | 20 | >640 | <5 | 40 | <5 | 10 |
| | 8546 | 40 | 40 | 10 | 20 | 160 | 20 | >640 | <5 | <5 | 5 | 10 |
| | 9506 | 5 | 5 | <5 | 10 | 40 | 40 | >640 | <5 | <5 | <5 | 20 |
| 9 kD | 1728 | <5 | 5 | <5 | 5 | 10 | <5 | <5 | <5 | <5 | <5 | 10 |
| | 7430 | 5 | <5 | <5 | <5 | 40 | 10 | <5 | <5 | 5 | 10 | 10 |
| | 8116 | <5 | 80 | 5 | 5 | 10 | 5 | <5 | <5 | <5 | <5 | <5 |
| | 8783 | 20 | 80 | 20 | 20 | 40 | 40 | <5 | <5 | 5 | 5 | 10 |
| 47 kD | 2968 | 10 | 20 | <5 | <5 | 20 | 10 | <5 | <5 | >640 | 5 | 5 |
| | 2969 | 160 | 80 | 40 | 40 | 160 | 10 | <5 | <5 | >640 | 5 | 10 |
| | 3847 | <5 | 5 | <5 | <5 | 10 | <5 | <5 | <5 | >640 | 5 | 5 |
| | 3911 | 10 | 5 | <5 | 5 | 10 | 5 | 10 | <5 | >640 | 5 | 10 |
| Hsp70 | 7020 | 20 | 10 | 10 | 5 | 80 | 10 | 5 | <5 | 5 | >640 | <5 |
| | 7049 | 10 | 20 | 20 | 10 | 20 | 5 | <5 | <5 | 10 | >640 | <5 |
| Hsp65 | 3105 | 10 | 10 | 10 | 5 | 40 | 40 | <5 | <5 | 20 | 10 | >640 |
| | 9505 | 5 | 40 | 20 | 5 | 40 | 80 | <5 | <5 | <5 | 5 | >640 |
| Adjuvant | 1727 | <5 | <5 | <5 | <5 | 10 | 10 | <5 | <5 | <5 | <5 | <5 |
| | 3848 | 10 | 10 | 10 | 20 | 10 | 5 | <5 | <5 | <5 | <5 | <5 |
| | 7429 | | | | | | | | | | | |
| None | 8354 | 20 | 80 | 5 | 5 | 10 | 5 | <5 | <5 | <5 | <5 | >5 |
| | 8738 | <5 | 20 | <5 | 10 | 5 | <5 | <5 | <5 | <5 | 10 | >5 |

TABLE 3

| | | Serum titers at OD = 1.0 (day 178) | | | | | | | | | | |
|---|---|---|---|---|---|---|---|---|---|---|---|---|
| | | B854 | | | | | | | | | | |
| | | Form | 5255 | 3 + 5/C | 316F | C | AN5 | | | | | |
| | | Cells | USO | John. | Excr. | KCL | KCL | 14 kD | 9 kD | 47 kD | Hsp70 | Hsp65 |
| Paratb | 840 | >640 | >640 | >640 | >640 | >640 | >640 | 5 | 5 | 10 | 320 | 20 |
| | 6431 | >640 | >640 | >640 | >640 | <640 | >640 | <5 | 10 | 5 | 40 | 20 |
| | 7029 | >640 | >640 | >640 | >640 | >640 | >640 | <5 | 5 | 5 | 10 | 5 |
| | 9293 | >640 | >640 | >640 | >640 | >640 | >640 | <5 | 10 | 10 | 20 | 40 |
| 14 kD | 5955 | 20 | 80 | <5 | 20 | 320 | 5 | >640 | 5 | 20 | 10 | 40 |
| | 7002 | <5 | 20 | <5 | <5 | 320 | <5 | >640 | 10 | 10 | <5 | 20 |
| | 8546 | 10 | 80 | 5 | 20 | 80 | 10 | >640 | 40 | 5 | 5 | 20 |
| | 9506 | 5 | 40 | <5 | 10 | 160 | 5 | >640 | 20 | 10 | 10 | 20 |
| 9 kD | 1728 | <5 | 10 | <5 | 20 | 80 | <5 | 10 | <5 | 40 | 10 | 80 |
| | 7430 | <5 | 40 | <5 | 5 | 80 | 20 | 5 | 160 | 20 | <5 | 40 |
| | 8116 | <5 | 20 | <5 | <5 | 160 | 40 | 40 | 160 | 80 | 10 | 80 |
| | 8783 | 40 | 80 | 20 | 80 | 320 | 10 | 10 | 160 | 10 | 5 | 40 |
| 47 kD | 2968 | 40 | 80 | 10 | 10 | 40 | <5 | 10 | <5 | >640 | 20 | 40 |
| | 2969 | 20 | 80 | 10 | 40 | 320 | 5 | <5 | <5 | >640 | 5 | 20 |
| | 3847 | <5 | 80 | <5 | <5 | 80 | <5 | <5 | <5 | >640 | 10 | 10 |
| | 3911 | 80 | 160 | 20 | 10 | 80 | 5 | 40 | 20 | >640 | 10 | 40 |
| Hsp70 | 7020 | >640 | 80 | 80 | 10 | >640 | 40 | <5 | <5 | 20 | >640 | 5 |
| | 7049 | >640 | 640 | >640 | 10 | >640 | 320 | <5 | <5 | 80 | >640 | 10 |
| Hsp65 | 3105 | 320 | 80 | 160 | 20 | 80 | 40 | 10 | 20 | 40 | 40 | >640 |
| | 9505 | >640 | 160 | >640 | 20 | 80 | 80 | 80 | 320 | 160 | 80 | >640 |
| Adjuvant | 1727 | <5 | 20 | <5 | <5 | 20 | <5 | 5 | <5 | <5 | <5 | <5 |
| | 3848 | 10 | 80 | 10 | 10 | 20 | 5 | <5 | <5 | <5 | <5 | <5 |
| | 7429 | <5 | 20 | <5 | <5 | 40 | 10 | <5 | <5 | <5 | 5 | 10 |
| None | 8354 | <5 | 80 | <5 | <5 | 20 | 10 | <5 | <5 | <5 | <5 | <5 |
| | 8738 | <5 | 20 | <5 | <5 | 80 | <5 | <5 | <5 | <5 | <5 | <5 |

c) DTH Reactivity.

Delayed type hypersensitivity (DTH) reactivity was done according to EU-directive 64/432 (as amended by directive 97/12 and 98/46). Briefly, 2000 IE aviary PPD, and 2000 and 5000 IE bovine PPD were injected and the increase in skin thickness after 72 hours detected. An increase of more than 2 mm is regarded as a positive DTH response. In the whole cell killed paratuberculosis vaccine group, bovine PPD reactivity was detected in all four animals both after prime and boost immunisation (Table 4). Bovine PPD reactivity was detected in animals vaccinated with the 14 kD protein (¼) and Hsp65 (½) after prime immunisation, and the 47 kD protein (¼) after boost immunisation (Table 4). Bovine PPD reactivity was not detected in the groups immunised with the 9 kD protein and the 70 kD protein (Table 4).

Results: the 9 kD and Hsp70 proteins do not give a response in a DTH PPD test. In case a vaccine is needed that does contribute to protection against infection and additionally does not cross-react with PPD in a DTH PPD test, the novel 9 kD protein according to the present invention would be the vaccine component of choice, preferably in combination with Hsp70.

TABLE 4
DTH reactivity

| | | DTH | | DTH | | | DTH | | |
|---|---|---|---|---|---|---|---|---|---|
| | | (day 56) | | (day 52) | | | (day 178) | | |
| | | A2 k | B2 K | A2 k | B2 k | B5 k | A2 K | B2 K | B5 K |
| Ptb | 840 | 0 | 0 | 23 | 6 | 9 | 12 | 2 | 8 |
| | 6431 | 0 | 0 | 25 | 15 | 23 | 6 | 3 | 5 |
| | 7019 | 0 | 0 | 11 | 5 | 7 | 10 | 4 | 4 |
| | 9293 | 0 | 0 | 17 | 8 | 11.5 | 6 | 6 | 6 |
| 14 kD | 5955 | 0 | 0 | 0.5 | 1.0 | 0 | 0 | 0 | 0 |
| | 7002 | 0 | 0 | 2.5 | 0 | 0 | 0 | 0 | 0 |
| | 8546 | 0 | 0 | 1.5 | 0 | 4.5 | 0 | 0 | 0 |
| | 9506 | 0 | 0 | 1.0 | 0 | 1.5 | 5 | 0 | 0 |
| 9 kD | 1728 | 0 | 0 | 0 | 0 | 0 | 0 | 0 | 0 |
| | 7430 | 0 | 0 | 0 | 0 | 0 | 0 | 0 | 0 |
| | 8116 | 0 | 0 | 0 | 0 | 0 | 0 | 0 | 0 |
| | 8783 | 0 | 0 | 0 | 0 | 0 | 0 | 0 | 0 |
| 47 kD | 2968 | 0 | 0 | 0 | 0 | 0 | 0 | 0 | 0 |
| | 2969 | 0 | 0 | 0 | 0 | 0 | 1 | 0 | 0 |
| | 3847 | 0 | 0 | 0 | 0 | 0 | 0 | 0 | 0 |
| | 3911 | 0 | 0 | 0 | 0 | 0 | 1 | 0 | 4 |
| Hsp70 | 7020 | 0 | 0 | 1.5 | 0 | 0 | 0 | 0 | 0 |
| | 7049 | 0 | 0 | 0 | 0 | 0 | 0 | 0 | 0 |
| Hsp65 | 3105 | 0 | 0 | 10 | 0.5 | 6 | 2 | 0 | 0 |
| | 9505 | 0 | 0 | 0 | 0 | 0 | 0 | 0 | 0 |
| Adju | 1727 | 0 | 0 | 0 | 0 | 0 | 0 | 0 | 0 |
| | 3848 | 0 | 0 | 2.0 | 0.5 | 0 | 1 | 0 | 0 |
| | 7429 | 0 | 0 | 0 | 0 | 0 | 0 | 0 | 0 |
| None | 8354 | 2.7 | 0 | 2.5 | 0 | 0 | NT | NT | NT |
| | 8738 | 0 | 0 | 0 | 0 | 0 | 0 | 0 | 0 |

ΔDTH >2 mm is indicated in bold.
NT, Not tested; animal removed during experiment.

### LEGEND TO THE FIGURES

FIG. 1.A shows in a Western blot of a 2D-gel the presence of a 33 kD protein having a pI between 4.20 and 4.75 and visible as a horizontal row of about 3 spots.

FIG. 1.B shows in a Western blot of a 2D-gel the presence of a 60 kD protein having a pI between 5.60 and 6.15 and visible as a horizontal row of about 5 spots.

FIG. 1.C shows the 2D-gel stained with Coomasie Brillant Blue, in which the specific spots of both the 33 kD and the 60 kD protein are visible.

FIG. 1.D shows the 2D-gel stained with silver staining, in which the specific spots of both the 33 kD and the 60 kD protein are again visible.

FIG. 2.

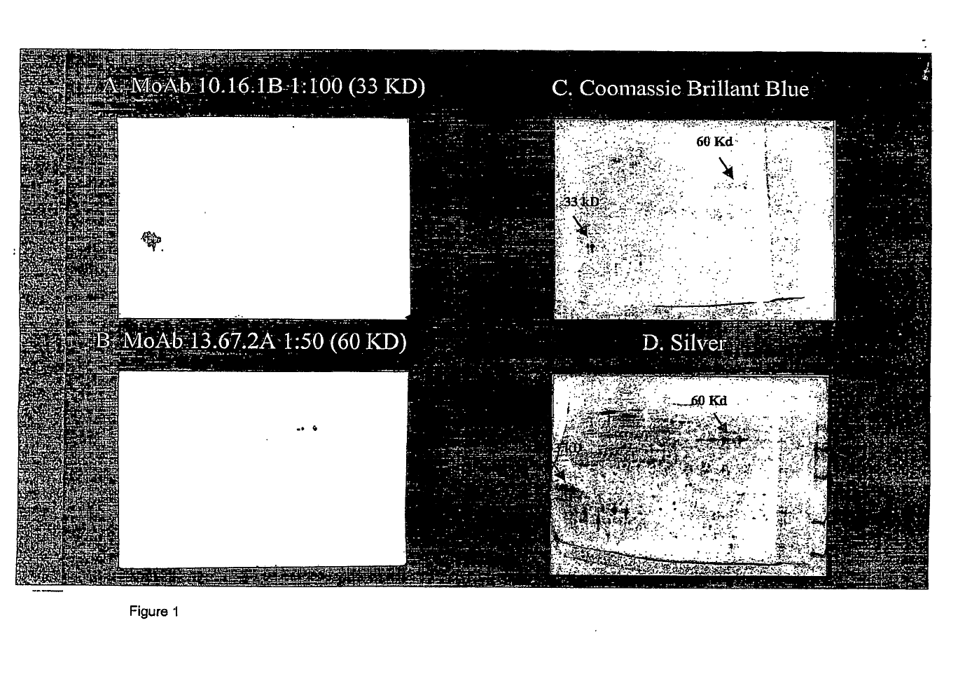

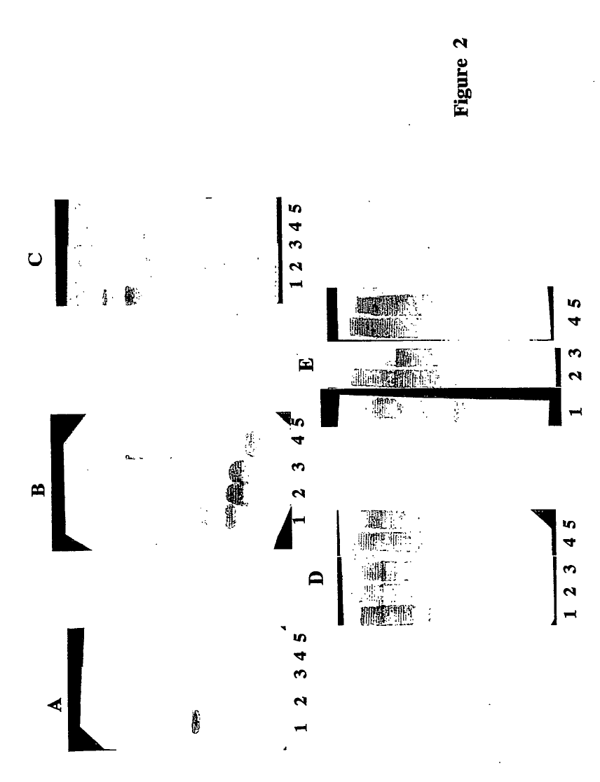

Immunoblots with serum samples (day 178) from animals immunised with recombinant purified 14 kD protein (panel A), with recombinant purified 9 kD protein (Panel B), with recombinant 47 kd (panel C), with recombinant purified 70 kD (panel D), and with recombinant 65 kD (panel E).

Lane 1, Recombinant purified protein of 14 (A), 9 (B), 47 (C), 70 (D) or 65 kD (E).

Lane 2, M. bovis strain AN5 whole cell sonicate.

Lane 3, M. avium paratuberculosis strain B854 whole cell sonicate.

Lane 4, M. avium paratuberculosis strains 3+5/C derived PPD.

Lane 5, M. bovis strain AN5 derived PPD.

1. A nucleic acid sequence encoding a 9 kD Mycobacterium avium subspecies paratuberculosis protein gene as depicted in SEQ ID NO: 5:

2. A nucleic acid sequence encoding a 14 kD Mycobacterium avium subspecies paratuberculosis protein gene as depicted in SEQ ID NO: 3.

3. A nucleic acid sequence encoding a 28 kD Mycobacterium avium subspecies paratuberculosis protein gene as depicted in SEQ ID NO: 1.

4. A nucleic acid sequence encoding a 47 kD Mycobacterium avium subspecies paratuberculosis protein gene as depicted in SEQ ID NO: 7.

5. A nucleic acid sequence encoding a Mycobacterium avium subspecies paratuberculosis protein gene as depicted in SEQ ID NO: 9.

6. A nucleic acid sequence encoding a Mycobacterium avium subspecies paratuberculosis protein gene as depicted in SEQ ID NO: 11.

7. A nucleic acid sequence encoding a Mycobacterium avium subspecies paratuberculosis protein gene as depicted in SEQ ID NO: 13.

8. A nucleic acid sequence encoding a Mycobacterium avium subspecies paratuberculosis protein gene as depicted in SEQ ID NO: 15.

9. A nucleic acid sequence encoding a Mycobacterium avium subspecies paratuberculosis protein gene as depicted in SEQ ID NO: 17.

10. A nucleic acid sequence encoding a 60 kD Mycobacterium avium subspecies paratuberculosis protein having a pI of 5.60-6.15.

11. A nucleic acid sequence encoding a 33 kD Mycobacterium avium subspecies paratuberculosis protein having a pI of 4.20-4.75.

12. A DNA fragment comprising a nucleic acid sequence according to claim 1.

13. A recombinant DNA molecule, comprising:
a nucleic acid sequence according to claim 1 under the control of a functionally linked promoter.

14. A live recombinant carrier, comprising:
a nucleic acid sequence according to claim 1.

15. A host cell, comprising:
a nucleic acid sequence according to claim 1.

16. A 9 kD Mycobacterium avium subspecies paratuberculosis protein or an immunogenic fragment of said protein, wherein said protein or immunogenic fragment thereof has a sequence homology of at least 90% having the amino acid sequence as depicted in SEQ ID NO: 6.

17. A 14 kD Mycobacterium avium subspecies paratuberculosis protein having the amino acid sequence as depicted in SEQ ID NO: 4.

18. A 28 kD Mycobacterium avium subspecies paratuberculosis protein having the amino acid sequence as depicted in SEQ ID NO: 2.

19. A 47 kD Mycobacterium avium subspecies paratuberculosis protein having the amino acid sequence as depicted in SEQ ID NO: 8.

20. A Mycobacterium avium subspecies paratuberculosis protein having the amino acid sequence as depicted in SEQ ID NO: 10.

21. A Mycobacterium avium subspecies paratuberculosis protein having the amino acid sequence as depicted in SEQ ID NO: 12.

22. A Mycobacterium avium subspecies paratuberculosis protein having the amino acid sequence as depicted in SEQ ID NO: 14.

23. A Mycobacterium avium subspecies paratuberculosis protein having the amino acid sequence as depicted in SEQ ID NO: 16.

24. A Mycobacterium avium subspecies paratuberculosis protein having the amino acid sequence as depicted in SEQ ID NO: 18.

25. A Mycobacterium avium subspecies paratuberculosis 60 kD protein having a pI of 5.60-6.15.

26. A Mycobacterium avium subspecies paratuberculosis 33 kD protein having a pI of 4.20-4.75.

27. (canceled)

28. (canceled)

29. (canceled)

30. A vaccine for combating Mycobacterium avium subspecies paratuberculosis infection, comprising:
at least one Mycobacterium avium subspecies paratuberculosis protein according to claim 16
a pharmaceutically acceptable carrier.

31. A vaccine for combating Mycobacterium avium subspecies paratuberculosis infection, comprising:
a nucleic acid sequence according to claim 1 and
a pharmaceutically acceptable carrier.

32. A vaccine for combating Mycobacterium avium subspecies paratuberculosis infection, comprising:
antibodies against a protein according to claim 16 and
a pharmaceutically acceptable carrier.

33. The vaccine according to claim 30, further comprising an adjuvant.

34. The vaccine according to claim 30, further comprising an additional antigen derived from a cattle pathogen, an antibody against said antigen or genetic information encoding said antigen.

35. The vaccine according to claim 34, wherein said cattle pathogen is selected from the group consisting of Bovine Herpesvirus, bovine Viral Diarrhoea virus, Parainfluenza type 3 virus, Bovine Paramyxovirus, Foot and Mouth Disease virus, Pasteurella haemolytica, Bovine Respiratory Syncytial Virus, Theileria sp., Babesia sp., Trypanosoma species, Anaplasma sp., Neospora caninum, Staphylococcus aureus, Streptococcus agalactiae, Mycoplasma, E. coli, Enterobacter, Klebsiella, Citrobacter and Streptococcus dysgalactiae.

36. A method for the preparation of a vaccine according to claim 30, comprising:
admixing a nucleic acid sequence according to claim 1 and
a pharmaceutically acceptable carrier.

37. A diagnostic kit, comprising:
suitable detection means and
a nucleic acid sequence according to claim 1 a primer thereof.


SEQUENCE LISTING (22 sequences)

SEQ ID NO: 1 - DNA, 1175 bp, mycobacterium avium paratuberculosis
  CDS (134)..(1144)
aattgcctcacgattcaatatcaccactctagtaataggattcccactcgtaccatcgactgtgtgtgattcctgccagacagcatcggcggggcgcgccgacacaacacatagtcagatagaggagacttccgtgccgaaccgacgccgacgcaagctttcgacagccatgagcgcggtcgccgccctggcagtggcgagtccttgcgcatacttccttgtctacgaatcgacggccggcaacaaggcgcccgagcaccacgagttcaagcaggccgcagtgatgagcgatctgccgggcgagctgatgggtgcgctgtcgcagggcctgtcgcagtttgggatcaacctgcccccggtgcccgccctgagcggcggcgccaccagcactcccggtctggccagccccggcctgggtagccccggcctgggcacgcccggcctgggaacgccgggcctgaccaatcccggtctgacgagccccggtgcgaccagtcccggcctgaccagtcccggcctgaccagtcctggtttgaccagccccggtctgaccagcccgggtgcggcgccgacgacgcccgggctcaccgcgcccggcgcgctgccgaccacgccgggcggcggggtcgccacccccggcgccgggctcaaccccgcgctgtccaaccccgggctgaccagcccggccgggacggcgccggggctgggcagcccgaccgtggcgccgagtgaggtgccgatcgactccggggccggcctggacccgggcgccggtggcacgtacccgatcctgggcgacccgtcgaccttcggtaacgcctcgccgatcggcggcggtggcaccggtctgggcggcggctcgagctcgggtggcagcggcggcctggtcaacgacgtgatgcaagccgccaaccagctcggcgcgggtcaggcgatcgacctgctcaagggcctggtgatgccggcgatcacgcagggcatgcacggcggcgcggccgcgggtgctttgcccggcgcggccggtgctctgcccggcgcggccggcgccctgcccggtgcggccggcgccctgccgggtgcggcgggcgccgcgggtgcgttgccggcggccgccggcgccgcgccggcactgcccccggtctagaccttttccaaaccatccaccagacggcacc

SEQ ID NO: 2 - protein, 336 aa, mycobacterium avium paratuberculosis
VPNRRRRKLSTAMSAVAALAVASPCAYFLVYESTAGNKAPEHHEFKQAAVMSDLPGELMGALSQGLSQFGINLPPVPALSGGATSTPGLASPGLGSPGLGTPGLGTPGLTNPGLTSPGATSPGLTSPGLTSPGLTSPGLTSPGAAPTTPGLTAPGALPTTPGGGVATPGAGLNPALSNPGLTSPAGTAPGLGSPTVAPSEVPIDSGAGLDPGAGGTYPILGDPSTFGNASPIGGGGTGLGGGSSSGGSGGLVNDVMQAANQLGAGQAIDLLKGLVMPAITQGMHGGAAAGALPGAAGALPGAAGALPGAAGALPGAAGAAGALPAAAGAAPALPPV

SEQ ID NO: 3 - DNA, 600 bp, mycobacterium avium paratuberculosis
  CDS (67)..(567)
ttcgagaagggatagcaggcggggccgggcggtgaacccgggaggcgcgcggtgcgtcttcagggcatgtcccgtttgtcatttgtctgcaggcttttggccgcaaccgctttcgccgtcgccctgctactcgggctgggcgacgtgccgcgcgcggcggccaccgacgaccgcctgcaattcaccgcgaccacgctcagcggcgcgccgttcaacggcgccagtctgcagggcaagcccgccgtgctgtggttctggacgccgtggtgcccgtactgcaacgccgaggccccgggcgtgagccgggtggccgccgccaacccgggcgtcaccttcgtcggcgtcgccgcccactccgaagtcggcgccatggccaacttcgtctccaagtacaacctgaacttcaccacgctcaacgacgccgacggcgcgatctgggcccgctacggcgtgccctggcagcccgcgtacgtgttctaccgggcggacggcagctccaccttcgtcaacaaccccacctcggcgatgccccaggacgaactggccgcccgggtggcggcgctgcgctgacgtggaccgcggtctggtcgggctggcggt

SEQ ID NO: 4 - protein, 167 aa, mycobacterium avium paratuberculosis
MSRLSFVCRLLAATAFAVALLLGLGDVPRAAATDDRLQFTATTLSGAPFNGASLQGKPAVLWFWTPWCPYCNAEAPGVSRVAAANPGVTFVGVAAHSEVGAMANFVSKYNLNFTTLNDADGAIWARYGVPWQPAYVFYRADGSSTFVNNPTSAMPQDELAARVAALR

SEQ ID NO: 5 - DNA, 366 bp, mycobacterium avium paratuberculosis
  CDS (34)..(366)
tagcggtgcattgactggggaaggtgtccacacatgaggctgtcgttgagcaaattgggcgttgcggtgggcagcgcggcagtggcattgaccgccgcggccggtgtcgcatccgccgaccccatggacgcgatcatcaacaccacctgcaactacgggcaggtgatcgccgcgctgaacgcgtccgacccggcggctgcccagcagctgaactcgtcgccgatggcgcagtcctacatccagcggttcctggcctccccgccggcgaagcgtcagcagatggcccagcagatccagggcatgccggccgcgcagcagtacatcaacgacatcaaccaggtcgcggtcacctgtaacaacttctga

SEQ ID NO: 6 - protein, 110 aa, mycobacterium avium paratuberculosis
MRLSLSKLGVAVGSAAVALTAAAGVASADPMDAIINTTCNYGQVIAALNASDPAAAQQLNSSPMAQSYIQRFLASPPAKRQQMAQQIQGMPAAQQYINDINQVAVTCNNF

SEQ ID NO: 7 - DNA, 1410 bp, mycobacterium avium paratuberculosis
  CDS (46)..(1410)
ctataggcataccccgacgcagaaacaacacggaaggtagctccggtggctccgaaggtctcgtccgatctgttctcgcagattgtcaattccggtcctggatcgtttctcgccaagcagctcggcgtcccgcaacccgagacgctgcgccgctaccggcccggtgacccgccgctggccgggtcgctgctgatcggcggcgagggccgcgtggtcgagccgctgcgggcggcgctggccaaggactacgacctggtcggcaacaacctgggcgggcgctgggccgaccggttcggcgggctggtcttcgacgccaccgggatcaccaccccggagggcctgaaggggctgtacgagttcttcaccccactgctgcgcaacctgggtcactgcgcccgcgtggtggtggtcggcaccacgcccgacgccgccgccggcccgcacgagcggatcgcccagcgcgccctggagggcttcacccggtcattgggcaaggagctgcgcaacggctcgacggtggcgctggtgtacctgtcgccggccgccaaacccgccgcgacgggcctggagtcgaccatgcggttcatcctgtcggccaagtccgcctacgtcgacggccaggtcttctacgtcggcgaggccgactccacccccccggcggactgggaacggccgctggacggcaaggtcgccatcgtgaccggtgcggcccgcggaatcggcgccacgatcgccgaggtgttcgcccgcgacggcgcccgcgtggtcgcgatcgacgtggaatcggccgccgagacgctggccgagacggccagccgggtcggcggcaccgcgctgtggctcgacgtcaccgcccccgacgccgtcgacaagatcaccgagcacctgcgcgagcaccacggcggtcacgccgacatcctggtcaacaacgccgggatcacccgcgacaagctgctggccaacatggacgacgcgcgctgggacgccgtgttggccgtgaatctgcttgccccacttcgccttaccgaagggctggtgggcaacggcagcatcggcgaaggcggccgcatcgtcggcctttcgtcgatggccggcatcgcgggcaaccgcggccagaccaactacgccaccaccaaggcaggcatgatcggcctcacccaggcgctggcgccggagctctacgacaagggcatcaccatcaacgccgtcgcgccgggattcatcgagacccagatgacggccgccatcccgctggccacccgcgaggtggggcgccggatgaactcgctgctgcagggcgggcagccggtggacgtcgccgaaaccatcgcctacttcgccagcccggcgtcgaacgcggtgaccggcaacgtcatccgggtctgcggccaggcgatgctgggggcatga

SEQ ID NO: 8 - protein, 454 aa, mycobacterium avium paratuberculosis
VAPKVSSDLFSQIVNSGPGSFLAKQLGVPQPETLRRYRPGDPPLAGSLLIGGEGRVVEPLRAALAKDYDLVGNNLGGRWADRFGGLVFDATGITTPEGLKGLYEFFTPLLRNLGHCARVVVVGTTPDAAAGPHERIAQRALEGFTRSLGKELRNGSTVALVYLSPAAKPAATGLESTMRFILSAKSAYVDGQVFYVGEADSTPPADWERPLDGKVAIVTGAARGIGATIAEVFARDGARVVAIDVESAAETLAETASRVGGTALWLDVTAPDAVDKITEHLREHHGGHADILVNNAGITRDKLLANMDDARWDAVLAVNLLAPLRLTEGLVGNGSIGEGGRIVGLSSMAGIAGNRGQTNYATTKAGMIGLTQALAPELYDKGITINAVAPGFIETQMTAAIPLATREVGRRMNSLLQGGQPVDVAETIAYFASPASNAVTGNVIRVCGQAMLGA

SEQ ID NO: 9 - DNA, 625 bp, mycobacterium avium paratuberculosis
  CDS (179)..(625)
  misc_feature (592)..(592): n is a, c, g, or t
aattcgcgcatacccgtcactggtcacaacgccacatgctggtaggctgtggaatcgagggtcaatccggatcggaccccaacgtcgacttgtgggcgccaattcgcgggttttcgcccagcaagtcgacgttcggcgcgaatcggtgaggtgggcacaggtgaatgacgaagaggacatgctggtcgccacggtgcgggcgttcatcgaccgcgaggtcaaaccgaccgtgcgcgaggtggagcacgccgatgcctatcccgaggcgtggatcgagcagatgaagcggatcgggatctacgggctggcggtgcccgaggaatacggtggttcgccggtgtccatgccgtgctacgtgcgggtcaccgagcagctggcgcgcggctggatgagcctggccggggcgatgggcgggcacaccgtggtggccaagctgctaacgctgttcggcaccgaggaccasaagcgggcctacctgccgcggatggccagcggcgaaatccgggccaccatggcgttgaccgagcccsgcggcggctcggacctgcagaacatgtcgaccaccgcgctgcccgaccccgactccgacggnctggtggtcaacggggccaagacctgnatcaac

SEQ ID NO: 10 - protein, 149 aa, mycobacterium avium paratuberculosis
  misc_feature (95)..(95): The ′Xaa′ at location 95 stands for Gln, or His.
MLVATVRAFIDREVKPTVREVEHADAYPEAWIEQMKRIGIYGLAVPEEYGGSPVSMPCYVRVTEQLARGWMSLAGAMGGHTVVAKLLTLFGTEDXKRAYLPRMASGEIRATMALTEPXGGSDLQNMSTTALPDPDSDGLVVNGAKTXIN

SEQ ID NO: 11 - DNA, 241 bp, mycobacterium avium paratuberculosis
  CDS (147)..(239)
gtgggggcaagccaattacgttcgcatcgacccggcacaggcggtcgctcacgtcatcaacatgccgctcatccccgatgaggctcgaatgaccttgctacgcaggcgctgaacgcacgacgaaacggaccggaggtgaaagggacatgagccacgccgatcaactcgctcggacgcacctggcgcccgatcctgcggacctgtcgcgcctggtcgccggcacccaccacgacccgcacgg

SEQ ID NO: 12 - protein, 31 aa, mycobacterium avium paratuberculosis
MSHADQLARTHLAPDPADLSRLVAGTHHDPH

SEQ ID NO: 13 - DNA, 236 bp, mycobacterium avium paratuberculosis
  CDS (8)..(214)
ggacaccaacgtgaccggggtgtttctcaccgcccaggcggcggcccgggcgatgatgcggcagggccgcggcggcgccatcatcaccaccgcctcgatgtccgggcacatcatcaacgtcccgcagcaggtcggccactactgcgccagcaaggcggccgtgatccagctgaccaaggccatggccgtcgaattctgcaggatccgtcgactctagactcgagcaagcttatgca

SEQ ID NO: 14 - protein, 69 aa, mycobacterium avium paratuberculosis
NVTGVFLTAQAAARAMMRQGRGGAIITTASMSGHIINVPQQVGHYCASKAAVIQLTKAMAVEFCRIRRL

SEQ ID NO: 15 - DNA, 419 bp, mycobacterium avium paratuberculosis
  CDS (25)..(417)
  misc_feature (331)..(331): n is a, c, g, or t
cggccaccgcacccaggggaggccatgactcacaccaaggccggtcgtgccgcgtggccggccgcctgcgcggtcgtcctgtccgccgccgcgctgttgtgcgcggcagcggccgccgcggacgaagccgatgacgcgttcctcgccggcctggccaagggcgggatcaccatgttcgacgacgacgacgcgatcgccatgggccacagcgtgtgctcgagcatcgacgccaaccccaacgtgtcgatgctggcgctgcggctgaccaagcaaaccccgttgacgccgaagcaatccggctacttcatcggtctttcggtcgccagctacntgcccgcagtacaaggacgacgtcgacccctcgctgggctggctgatcccgccgccgctgatgtgangttgccggccggcatcggcgt

SEQ ID NO: 16 - protein, 131 aa, mycobacterium avium paratuberculosis
  misc_feature (103)..(103): The ′Xaa′ at location 103 stands for Met, Val, or Leu.
MTHTKAGRAAWPAACAVVLSAAALLCAAAAAADEADDAFLAGLAKGGITMFDDDDAIAMGHSVCSSIDANPNVSMLALRLTKQTPLTPKQSGYFIGLSVASYXPAVQGRRRPLAGLADPAAADVXLPAGIG

SEQ ID NO: 17 - DNA, 392 bp, mycobacterium avium paratuberculosis
  CDS (94)..(390)
cggcgtagcatcgtcaagtcgttgcccgcgctgatgccggagcggcagtaaggagttcggctggtgcaaaaacgcttgcccacagtcgttttggtgctgacggccgttgtcgccggtatcgccgggtgcagcgcggcgcagaccgtgccgcgcaaggccgcccggctgaccatcgacggtgccacccacacgacccgcccgccgtcctgccggcaggaccagatgtatcggaccatcaacatccccgaccacgacggtggagtcgaagcggtggtgctgctcagcggttaccgggtgatgccgcagtgggtgaagatccggaacgtcgacggcttcaccggcagtctactggccasggcggagtgggcgacgcgcacgtcgatctcacmaat

SEQ ID NO: 18 - protein, 99 aa, mycobacterium avium paratuberculosis
  misc_feature (88)..(88): The ′Xaa′ at location 88 stands for Arg, or Thr.
VLTAVVAGIAGCSAAQTVPRKAARLTIDGATHTTRPPSCRQDQMYRTINIPDHDGGVEAVVLLSGYRVMPQWVKIRNVDGFTGSLLAXAEWATRTSISX

SEQ ID NO: 19 - DNA, 1884 bp, mycobacterium avium paratuberculosis
  CDS (13)..(1884)
taaccaggagcaatggctcgtgcggtcggtatcgacctcgggaccaccaactccgtcgtcgcagtcctcgagggcggtgaccccgtcgtcgtcgccaactccgagggctcgcggaccaccccgtccatcgtcgcgttcgcccgcaacggcgaggtgctcgtcggccagcccgccaagaaccaggcggtgaccaacgtcgaccgcaccatccgttcggtcaagcggcacatgggcaccgactggtccatcgagatcgacggcaagaaatacaccgctcaggagatcagcgcccgcgtgctgatgaagctcaagcgcgacgccgaggcctatctgggtgaggacatcaccgacgcggtcatcaccgtaccggcgtacttcaacgacgcccagcgtcaggcgaccaaggaagccggccagatcgccggcctcaacgtgctgcgcatcgtcaacgagccgaccgcggccgcgctggcctacggcctggacaagggcgagaaggagcagaccatcctggtcttcgacctcggcggcggcacgttcgacgtttcgctgctcgagatcggcgagggtgtggtcgaggtccgcgccaccagcggtgacaaccaactcggtggcgacgactgggacgaccggatcgtcaactggctggtcgacaagttcaagggcaccagcggcatcgacctgaccaaggacaagatggccatgcagcggctgcgtgaggccgccgagaaggccaagatcgagttgtccagctcgcagagcacctcgatcaacctgccctacatcaccgtcgacgcggacaagaacccgctgttcctcgacgagcagctgacccgcgccgaattccagcgcatcacccaggatctgctggaccgcacccgtcagccgttcaagtcggtgatcgccgacgccggcatctcggtgtccgacatcgaccacgtggtgctggtgggtggttccacccggatgcccgcggtgaccgacctggtcaaggaactcaccggcggcaaggagcccaacaagggcgtcaaccccgacgaggttgtcgcggtgggtgccgccctgcaggccggtgtgcttaagggcgaggtgaaagacgttctgctgcttgacgttacgccgctgagcctgggtatcgagaccaagggtggcgtgatgaccaagctgatcgaacgcaacaccaccatcccgaccaagcggtccgagacgttcaccacggccgacgacaaccagccgtcggtgcagatccaggtgtatcagggtgagcgcgaaatcgccgcgcacaacaagctgctcggctccttcgagctgaccggaattccgccggcgccccgcggcgtgccgcagatcgaggtcaccttcgacatcgacgccaacggcatcgtgcacgtcaccgccaaggacaagggcaccggtaaggagaacacgatcaagatccaggagggctccggcctgtccaaggaggagatcgaccggatgatcaaggacgccgaggcgcacgccgaggaggaccgcaagaggcgcgaggaagccgacgtccgcaaccaagcggaatcgcttgtctaccagacggagaagttcgtcaaggaccagcgcgaggccgagggcggctcgaaggttcccgaggagacgctgtccaaggtcgacgccgcgatcgccgacgccaagacggccctgggcggcaccgacatcaccgcgatcaagtcggcgatggagaagctcggccaggagtcgcaagcgctgggacaggcaatctacgaggccacccaggccgagtccgcccaggctggcgggccggacggtgccgcggccggcggcgggtccggatccgccgacgatgttgtggacgcggaggtggtcgacgatgaccgggagtccaagtga

SEQ ID NO: 20 - protein, 623 aa, mycobacterium avium paratuberculosis
MARAVGIDLGTTNSVVAVLEGGDPVVVANSEGSRTTPSIVAFARNGEVLVGQPAKNQAVTNVDRTIRSVKRHMGTDWSIEIDGKKYTAQEISARVLMKLKRDAEAYLGEDITDAVITVPAYFNDAQRQATKEAGQIAGLNVLRIVNEPTAAALAYGLDKGEKEQTILVFDLGGGTFDVSLLEIGEGVVEVRATSGDNQLGGDDWDDRIVNWLVDKFKGTSGIDLTKDKMAMQRLREAAEKAKIELSSSQSTSINLPYITVDADKNPLFLDEQLTRAEFQRITQDLLDRTRQPFKSVIADAGISVSDIDHVVLVGGSTRMPAVTDLVKELTGGKEPNKGVNPDEVVAVGAALQAGVLKGEVKDVLLLDVTPLSLGIETKGGVMTKLIERNTTIPTKRSETFTTADDNQPSVQIQVYQGEREIAAHNKLLGSFELTGIPPAPRGVPQIEVTFDIDANGIVHVTAKDKGTGKENTIKIQEGSGLSKEEIDRMIKDAEAHAEEDRKRREEADVRNQAESLVYQTEKFVKDQREAEGGSKVPEETLSKVDAAIADAKTALGGTDITAIKSAMEKLGQESQALGQAIYEATQAESAQAGGPDGAAAGGGSGSADDVVDAEVVDDDRESK

SEQ ID NO: 21 - DNA, 1701 bp, mycobacterium avium paratuberculosis
  CDS (76)..(1701)
gcagcctggtcgtccgtcgcgggcactgcacccggccaggacgtgtcatccccaatccggaggaatcacttcgcaatggccaagacaattgcgtacgacgaagaggcccgtcgcggcctcgagcgggggctcaacgccctcgccgacgcggtaaaggtcacgttgggccccaagggtcgcaacgtcgtcctggagaagaagtggggtgcccccacgatcaccaacgatggtgtgtccatcgccaaggagatcgagctggaggacccgtacgagaagatcggcgccgagctggtcaaggaagtcgccaagaagaccgacgacgtcgccggtgacggcacgacgacggccacggtgctcgcccaggcgttggtccgcgagggcctgcgcaacgtcgcggccggcgccaacccgctgggtctcaagcgcggcatcgagaaggccgtcgagaaggtcaccgagaccctgctcaagtcggccaaggaggtcgagaccaaggaccagatcgctgccaccgcggccatctccgcgggcgaccagtcgatcggcgacctgatcgccgaggcgatggacaaggtcggcaacgagggcgtcatcaccgtcgaggagtccaacaccttcggcctgcagctcgagctcaccgagggtatgcggttcgacaagggttacatctcgggctacttcgtcacggacgccgagcgtcaggaagcggtcctcgaggacccgttcatcctgctggtcagctccaaggtctcgaccgtcaaggacctgctgccgctgctggagaaggtcatccaggccggcaagccgctgctgatcatcgccgaggacgtcgagggcgaggccctgtccaccctggtcgtcaacaagatccgcggcaccttcaagtcggtggccgtcaaggcgcccggcttcggcgaccgccgcaaggcgatgcttcaggacatggccatcctcaccggcggccaggtcatcagcgaagaggtcggcctgtcgctggagagcgccgacatctcgctgctcggtaaggcccgcaaggtcgtcgtcaccaaggacgagaccaccatcgtcgagggcgccggtgactccgacgccatcgccggccgggtggcccagatccgcaccgagatcgagaacagcgactccgactacgaccgcgagaagctgcaggagcggctggccaagctggccggcggcgtggcggtgatcaaggccggcgccgcgaccgaggtcgagctcaaggagcgcaagcaccgcatcgaggacgcggtccgcaacgccaaggcggccgtggaggagggcatcgtcgccggcggtggcgtggccctgctgcacgcgatcccggctctggacgagctgaagctcgagggcgaagaggcgaccggcgccaacatcgtccgggtggccctcgaggctccgctgaagcagatcgccttcaacggtggcctggagcccggcgtggtggccgagaaggtccgcaactcgcccgccggtaccggcctcaacgccgccaccggtgagtacgaggacctgctcaaggccggcattgccgacccggtgaaggtcacccgctcggcgctgcagaacgcggcgtccatcgcggggctgttcctgaccaccgaggcggtcgtcgccgacaagccggagaaggcggccgctcccgcgggcgacccgaccggcggcatgggcggcatggacttctga

SEQ ID NO: 22 - protein, 541 aa, mycobacterium avium paratuberculosis
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEKVTETLLKSAKEVETKDQIAATAAISAGDQSIGDLIAEAMDKVGNEGVITVEESNTFGLQLELTEGMRFDKGYISGYFVTDAERQEAVLEDPFILLVSSKVSTVKDLLPLLEKVIQAGKPLLIIAEDVEGEALSTLVVNKIRGTFKSVAVKAPGFGDRRKAMLQDMAILTGGQVISEEVGLSLESADISLLGKARKVVVTKDETTIVEGAGDSDAIAGRVAQIRTEIENSDSDYDREKLQERLAKLAGGVAVIKAGAATEVELKERKHRIEDAVRNAKAAVEEGIVAGGGVALLHAIPALDELKLEGEEATGANIVRVALEAPLKQIAFNGGLEPGVVAEKVRNSPAGTGLNAATGEYEDLLKAGIADPVKVTRSALQNAASIAGLFLTTEAVVADKPEKAAAPAGDPTGGMGGMDF